(19) United States
(12) Patent Application Publication
(10) Pub. No.: US 2025/0360071 A1
(43) Pub. Date: Nov. 27, 2025

(54) AGENT FOR INDUCING CILIUM

(71) Applicant: MED R&D Co. Ltd, Tsukuba-shi (JP)

(72) Inventors: Hiroko ISODA, Tsukuba-shi (JP); Mitsutoshi NAKAJIMA, Tsukuba-shi (JP); Masako SAITO, Tokyo (JP); Yuko KATAGIRI, Tokyo (JP)

(21) Appl. No.: 18/872,598

(22) PCT Filed: Jun. 9, 2023
(86) PCT No.: PCT/JP2023/021606

§ 371 Date: Dec. 6, 2024

(30) Foreign Application Priority Data
Jun. 10, 2022 (JP) 2022-094480

(51) Int. Cl. A61K 8/63; A61Q 7/00

(52) U.S. Cl. CPC A61K 8/63; A61Q 7/00; A61K 2800/92

(57) ABSTRACT

An oleanane-type triterpenoid or a glycoside thereof is useful as a compound having an effect of inducing formation of cilium and activating hair papilla cells or hair matrix cells.

### TECHNICAL FIELD

The present invention relates to an agent for inducing cilium. Priority is claimed on Japanese Patent Application No. 2022-094480, filed on Jun. 10, 2022, the content of which is incorporated herein by reference.

### BACKGROUND ART

Hair loss of the head is a symptom caused by various causes such as aging, and is not a dangerous symptom in itself. However, in consideration of the importance of the hair loss of the head on the appearance, various treatment methods have been studied since the hair loss can have a significant impact on the quality of life of an individual.

As a currently available method for treating alopecia, methods using synthetic chemical components are mainly known. For example, “minoxidil” and “finasteride” have been approved by the US Food and Drug Administration (FDA) as a treatment agent for alopecia.

In addition, Patent Document 1 proposes that a component including an extract of carrot of the Panax genus belongs to the Araliaceae family has a hair effect.

### CITATION LIST

### Patent Document

Patent Document 1: Japanese Unexamined Patent Application, First Publication No. 2019-026634

### SUMMARY OF INVENTION

### Technical Problem

However, the administration of an existing drug such as minoxidil may cause a number of severe side effects (impotence, dizziness, unintended hair growth, weakness, headache, skin rash, and the like). In addition, the therapeutic effect may remain temporarily.

Moreover, in the carrot extract described in Patent Document 1, there is no description of Examples that directly confirm whether any specific compound has a hair loss preventing effect and a hair growth promoting effect.

An object of the present invention is to provide a compound having an effect of inducing formation of cilium and activating hair papilla cells or hair matrix cells.

### Solution to Problem

The present invention includes the following aspects.

[1] An agent for inducing cilium including, as an active ingredient:
an oleanane-type triterpenoid or a glycoside thereof.
   [2] The agent for inducing cilium according to [1],
in which the oleanane-type triterpenoid or a glycoside thereof is a compound represented by Formula (A) or (B),

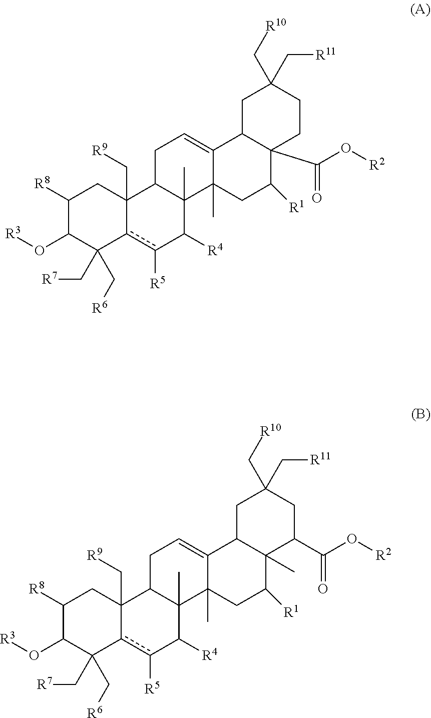

[in Formulae (A) and (B), R¹, R⁴, R⁵, R⁶, R⁷, R⁸, R⁹, R¹⁰, and R¹¹ each independently represent a hydrogen atom or a hydroxyl group, R² and R³ each independently represent a hydrogen atom or an oligosaccharide chain including 1 to 5 monosaccharide units, and [symbol]
represents a single bond or a double bond].
   [3] The agent for inducing cilium according to [2],
in which the oleanane-type triterpenoid or a glycoside thereof is a compound represented by Formula (1),

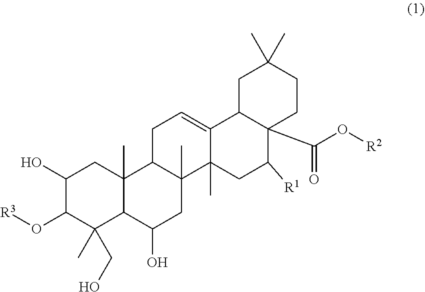

[in Formula (1), R¹, R², and R³ each have the same definitions as those in Formula (A) or (B)].
   [4] The agent for inducing cilium according to [2] or [3],
in which R² in Formula (A) or (B) is a group represented by any one of Formulae (2) to (7),

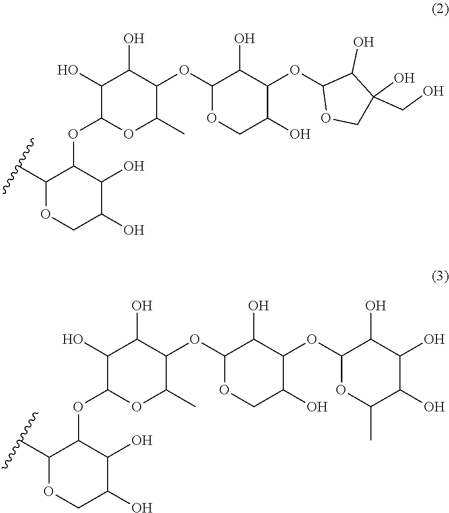 [SMILES: *C1OCC(O)C(O)C1OC1OC(C)C(OC2OCC(O)C(OC3OCC(O)(CO)C3O)C2O)C(O)C1O ; *C1OCC(O)C(O)C1OC1OC(C)C(OC2OCC(O)C(OC3OC(C)C(O)C(O)C3O)C2O)C(O)C1O]

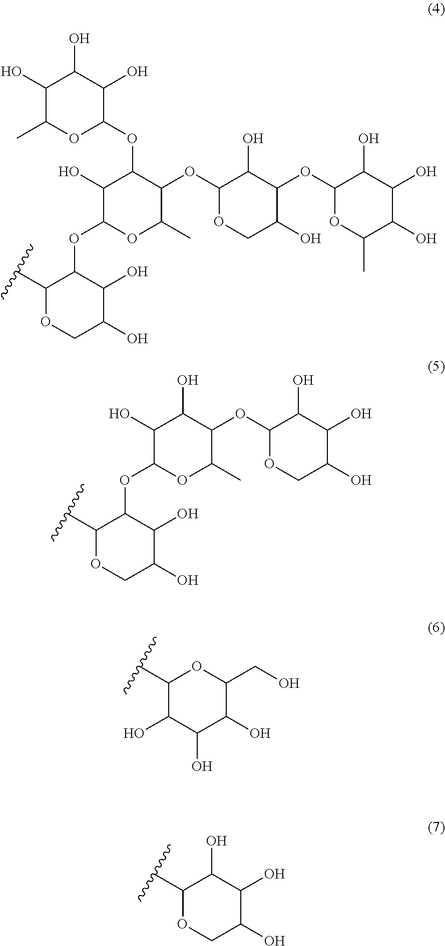 [SMILES: *C1OCC(O)C(O)C1OC1OC(C)C(OC2OCC(O)C(OC3OC(C)C(O)C(O)C3O)C2O)C(OC2OC(C)C(O)C(O)C2O)C1O ; *C1OCC(O)C(O)C1OC1OC(C)C(OC2OCC(O)C(O)C2O)C(O)C1O ; *C1OC(CO)C(O)C(O)C1O ; *C1OCC(O)C(O)C1O]

[5] The agent for inducing cilium according to any one of [2] to [4],
in which R³ in Formula (A) or (B) is a group represented by any one of Formulae (8) to (12),

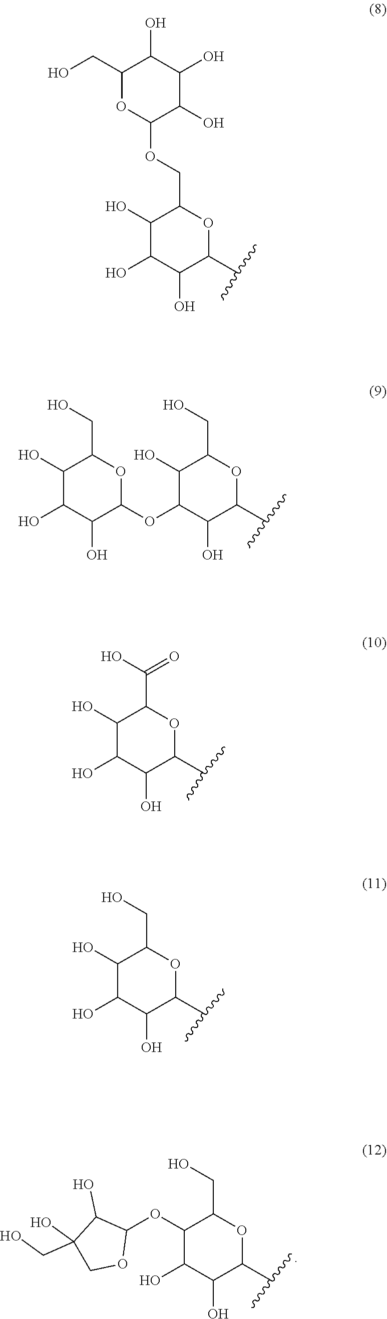 [SMILES: *C1OC(COC2OC(CO)C(O)C(O)C2O)C(O)C(O)C1O ; *C1OC(CO)C(O)C(OC2OC(CO)C(O)C(O)C2O)C1O ; *C1OC(C(=O)O)C(O)C(O)C1O ; *C1OC(CO)C(O)C(O)C1O ; *C1OC(CO)C(OC2OCC(O)(CO)C2O)C(O)C1O]

[6] The agent for inducing cilium according to any one of [2] to [5],
in which the compound represented by Formula (A) or (B) is a compound selected from the group consisting of maslinic acid (CAS number: 4373-41-5), oleanolic acid (CAS number: 508-02-1), 16α-hydroxyprotobassic acid (CAS number: 144223-48-3), protobassic acid (CAS number: 37905-13-8), arganine A (CAS number: 144425-20-7), arganine B (CAS number: 144425-21-8), arganine C (CAS number: 132023-46-2), arganine D (CAS number: 144442-85-3), arganine E (CAS number: 144442-86-4), arganine F (CAS number: 144425-22-9), arganine G (CAS number: 174630-13-8), arganine H (CAS number: 174630-14-9), arganine J (CAS number: 174630-15-0), tieghemelin (CAS number: 479072-94-1), butyroside B (CAS number: 144576-92-1), butyroside C (CAS number: 159803-58-4), butyroside D (CAS number: 159803-59-5), Mi-saponin A (CAS number: 54328-42-6), mimusopsin (CAS number: 171674-86-5), olean-12-en-28-oic acid, 3-[(3-O-β-D-glucopyranosyl-β-D-glucopyranosyl)oxy]-2,6,16,23-tetrahydroxy-, O-6-deoxy-α-L-mannopyranosyl-(1→3)-O-β-D-xylopyranosyl-(1→4)-O-6-deoxy-α-L-mannopyranosyl-(1→2)-α-L-arabinopyranosyl ester, (2β,3β,4α,6β,16α)-(9CI, ACI) (CAS number: 912967-77-2), olean-12-en-28-oic acid, 3-[(3-O-β-D-glucopyranosyl-β-D-glucopyranosyl)oxy]-2,6,16,23-tetrahydroxy-, O-6-deoxy-α-L-mannopyranosyl-(1→3)-O-β-D-xylopyranosyl-(1→4)-O-[6-deoxy-α-L-mannopyranosyl-(1→3)]-O-6-deoxy-α-L-mannopyranosyl-(1→2)-α-L-arabinopyranosyl ester, (2β,3β,4α,6β,16α)-(9CI) (CAS number: 451462-60-5), olean-12-en-28-oic acid, 3-[(3-O-β-D-glucopyranosyl-β-D-glucopyranosyl)oxy]-2,6,23-trihydroxy-, O-6-deoxy-α-L-mannopyranosyl-(1→3)-O-β-D-xylopyranosyl-(1→4)-O-[6-deoxy-α-L-mannopyranosyl-(1→3)]-O-6-deoxy-α-L-mannopyranosyl-(1→2)-α-L-arabinopyranosyl ester, (2β,3β,4α,6β)-(9CI) (CAS number: 451462-61-6), and a compound represented by Formula (P6),

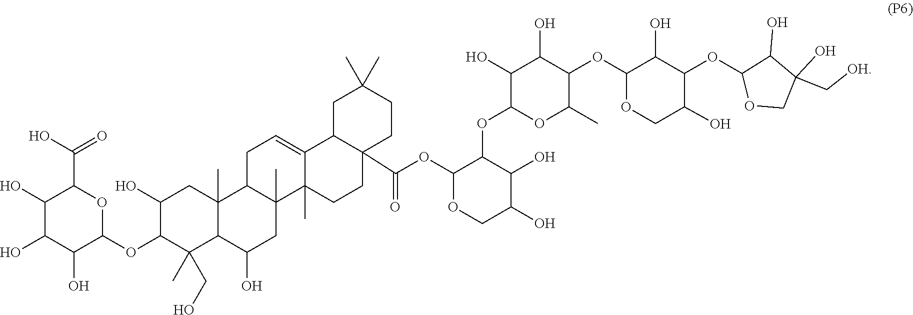

[7] The agent for inducing cilium according to any one of [2] to [6],
in which the compound represented by Formula (A) or (B) is a compound selected from the group consisting of butyroside D, arganine D, arganine E, tieghemelin, maslinic acid, and Mi-saponin A.
   [8] The agent for inducing cilium according to any one of [1] to [7],
in which the agent for inducing cilium is for oral administration.
   [9] A composition for inducing cilium, including:
the agent for inducing cilium according to any one of [1] to [7]; and a pharmaceutically acceptable carrier.
   [10] The composition for inducing cilium according to [9],
in which the composition is for oral administration.

It can also be said that the present invention includes the following aspects.

[P1] A hair growth agent including, as an active ingredient:
a compound represented by Formula (P1),

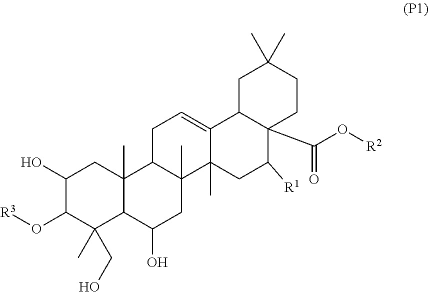

[in Formula (P1), R¹ is a hydrogen atom or a hydroxyl group, and R² and R³ are each independently an oligosaccharide chain including 1 to 5 monosaccharide units].
   [P2] The hair growth agent according to [P1],
in which R² in Formula (P1) is a group represented by Formula (P2) or (P3),

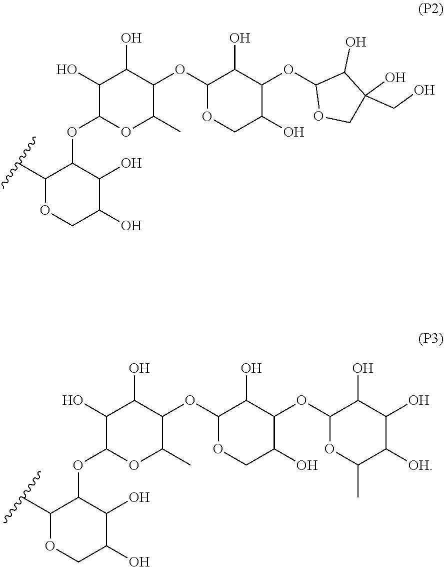 [SMILES: *C1OCC(O)C(O)C1OC1OC(C)C(OC2OCC(O)C(OC3OCC(O)(CO)C3O)C2O)C(O)C1O ; *C1OCC(O)C(O)C1OC1OC(C)C(OC2OCC(O)C(OC3OC(C)C(O)C(O)C3O)C2O)C(O)C1O]

[P3] The hair growth agent according to [P1] or [P2],
in which R³ in Formula (P1) is a group represented by Formula (P4) or (P5),

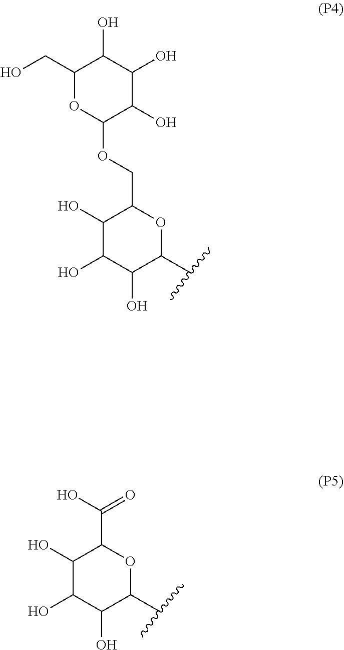 [SMILES: *C1OC(COC2OC(CO)C(O)C(O)C2O)C(O)C(O)C1O ; *C1OC(C(=O)O)C(O)C(O)C1O]

[P4] The hair growth agent according to any one of [P1] to [P3],
in which the compound represented by Formula (P1) is a compound selected from the group consisting of arganine A (CAS number: 144425-20-7), arganine B (CAS number: 144425-21-8), arganine D (CAS number: 144442-85-3), arganine E (CAS number: 144442-86-4), tieghemelin (CAS number: 479072-94-1), butyroside C (CAS number: 159803-58-4), butyroside D (CAS number: 159803-59-5), and a compound represented by Formula (P6),

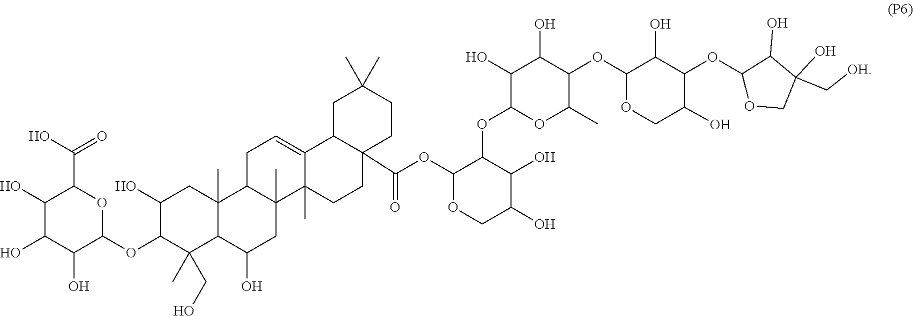

[P5] The hair growth agent according to any one of [P1] to [P4],
in which the compound represented by Formula (P1) is a compound selected from the group consisting of butyroside D, arganine D, and arganine E.
   [P6] The hair growth agent according to any one of [P1] to [P5],
in which the hair growth agent is for oral administration.
   [P7] A composition for hair growth, including:
the hair growth agent according to any one of [P1] to [P6]; and a pharmaceutically acceptable carrier.
   [P8] The composition for hair growth according to [P7],
in which the composition for hair growth is for oral administration.

### Advantageous Effects of Invention

According to the present invention, it is possible to provide a compound having an effect of activating IQCB1 to induce formation of cilium and activating hair papilla cells or hair matrix cells.

### BRIEF DESCRIPTION OF DESCRIPTION

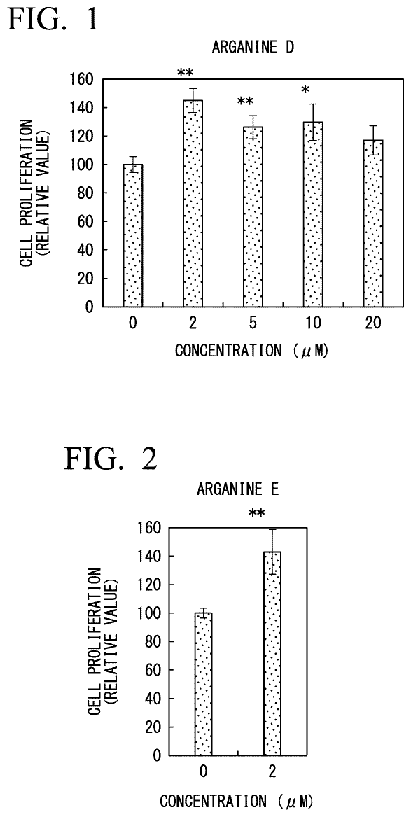

FIG. 1 A graph showing the results of examining the influence of arganine D on the proliferation of HFDPCs in Experimental Example 1.

FIG. 2 A graph showing the results of examining the influence of arganine E on the proliferation of HFDPCs in Experimental Example 1.

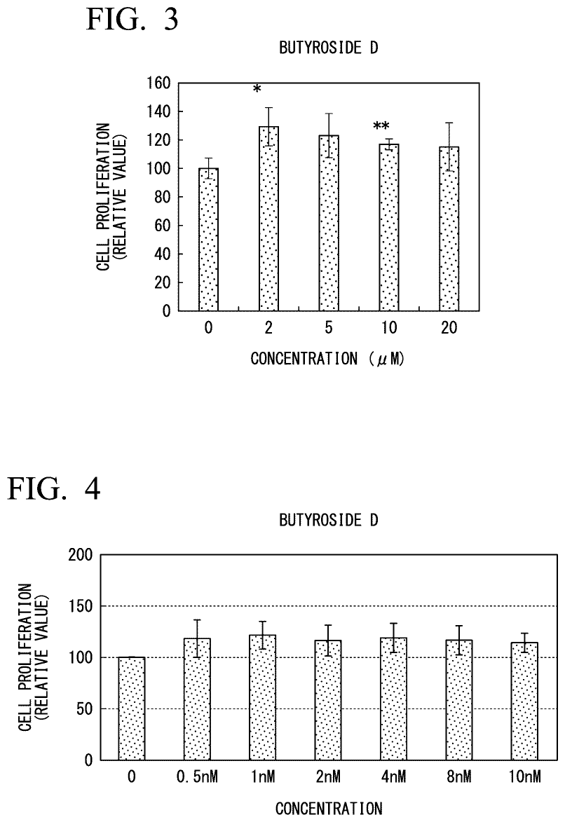

FIG. 3 A graph showing the results of examining the influence of butyroside D on the proliferation of HFDPCs in Experimental Example 1.

FIG. 4 A graph showing the results of examining the influence of butyroside D on the proliferation of HFDPCs in Experimental Example 2.

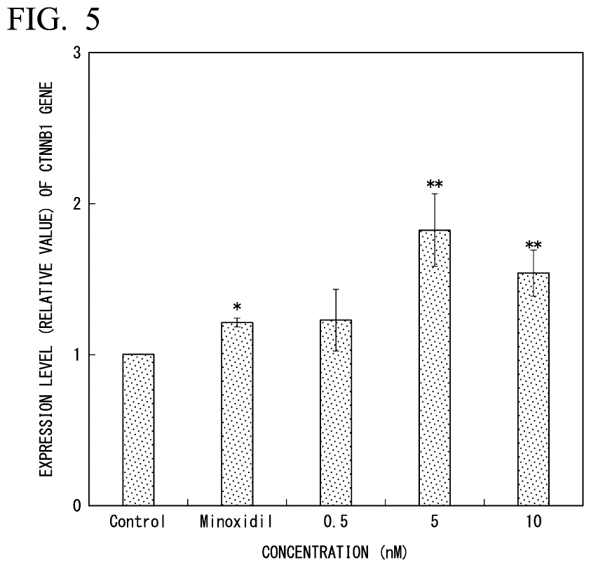

FIG. 5 A graph showing the results of quantitative real-time RT-PCR of a CTNNB1 gene in Experimental Example 3.

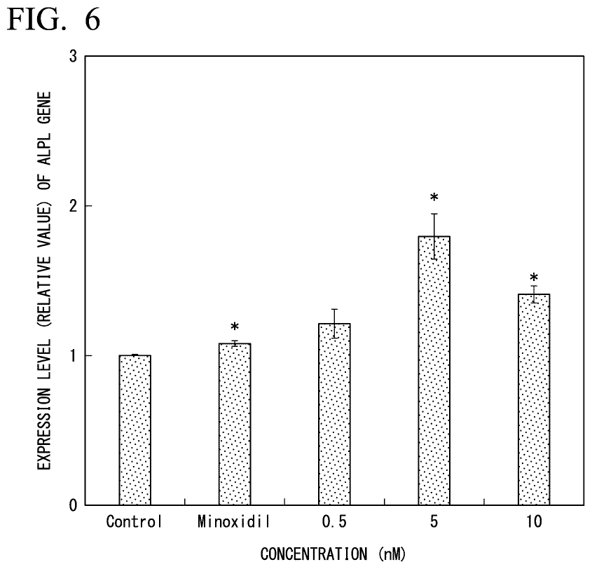

FIG. 6 A graph showing the results of quantitative real-time RT-PCR of an ALPL gene in Experimental Example 3.

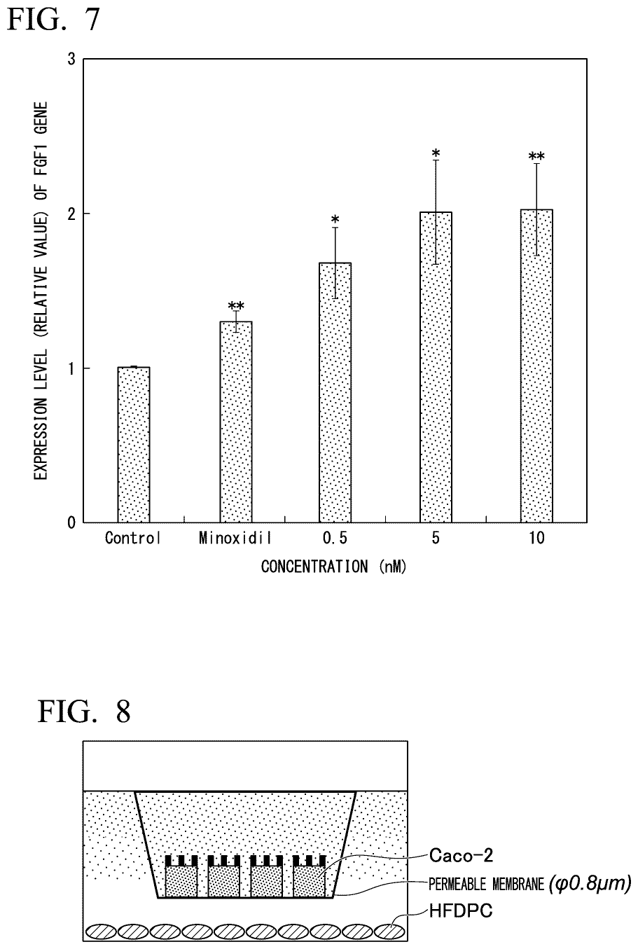

FIG. 7 A graph showing the results of quantitative real-time RT-PCR of an FGF1 gene in Experimental Example 3.

FIG. 8 A schematic view describing a co-culture system used in Experimental Example 4.

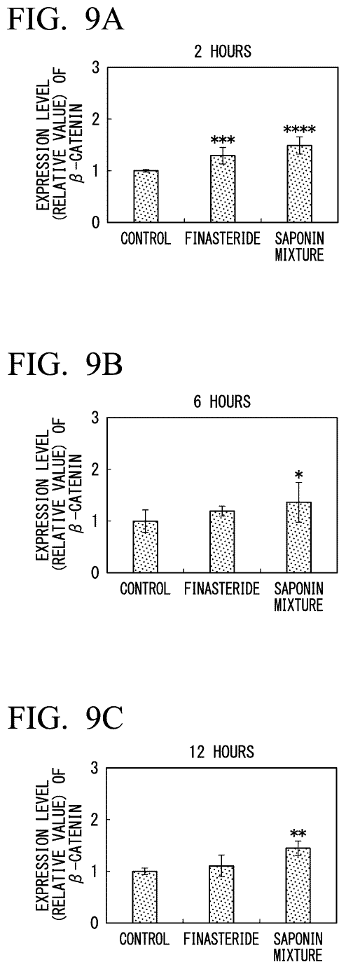

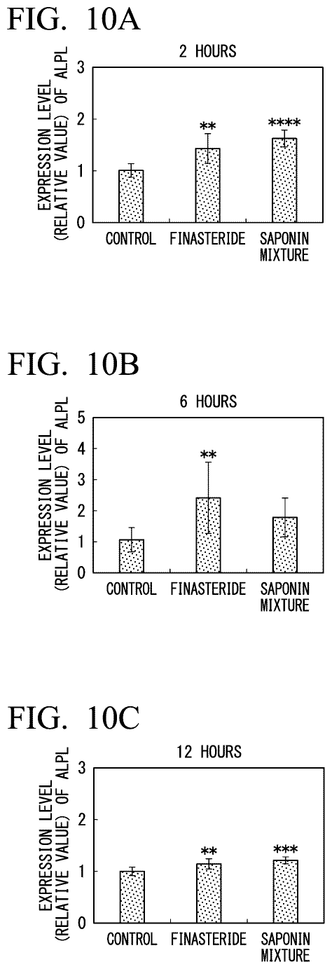

FIG. 9A A graph showing the measurement results of an expression level of a β-catenin gene in Experimental Example 4.

FIG. 9B A graph showing the measurement results of an expression level of the β-catenin gene in Experimental Example 4.

FIG. 9C A graph showing the measurement results of an expression level of the β-catenin gene in Experimental Example 4.

FIG. 10A A graph showing the measurement results of an expression level of an ALPL gene in Experimental Example 4.

FIG. 10B A graph showing the measurement results of an expression level of the ALPL gene in Experimental Example 4.

FIG. 10C A graph showing the measurement results of an expression level of the ALPL gene in Experimental Example 4.

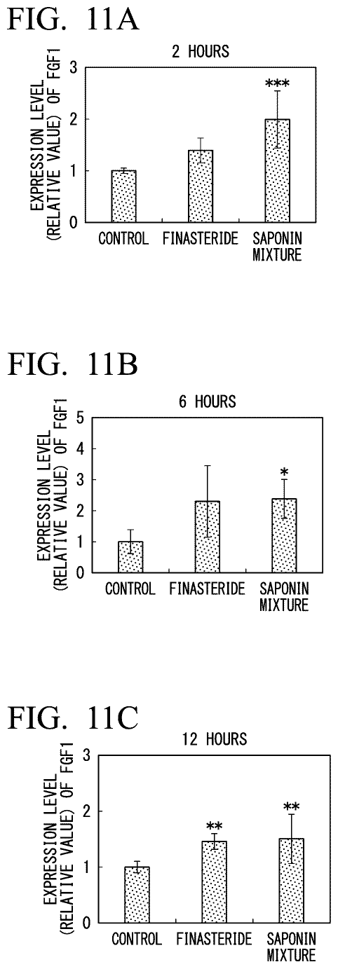

FIG. 11A A graph showing the measurement results of an expression level of an FGF1 gene in Experimental Example 4.

FIG. 11B A graph showing the measurement results of an expression level of the FGF1 gene in Experimental Example 4.

FIG. 11C A graph showing the measurement results of an expression level of the FGF1 gene in Experimental Example 4.

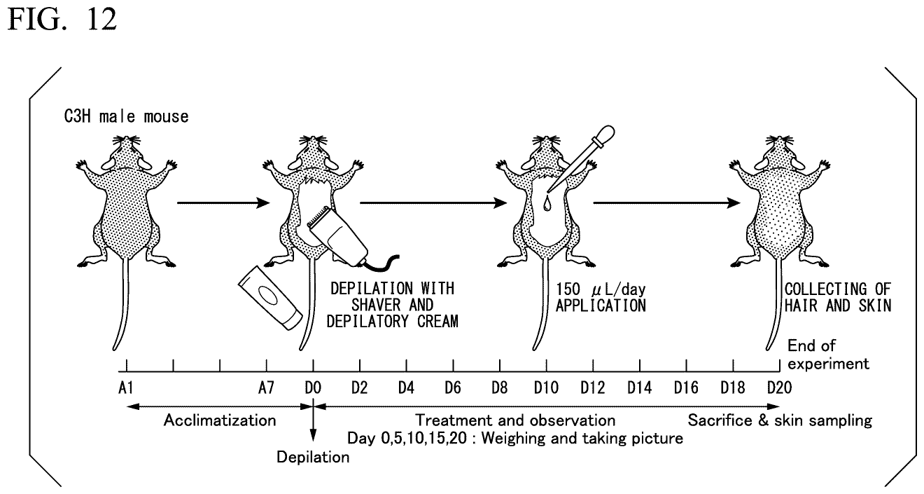

FIG. 12 A schematic view showing an experimental schedule of Experimental Example 5.

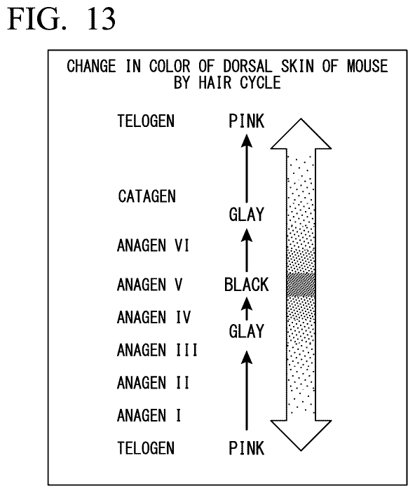

FIG. 13 A view showing a change in color of the dorsal skin of a mouse due to a hair cycle.

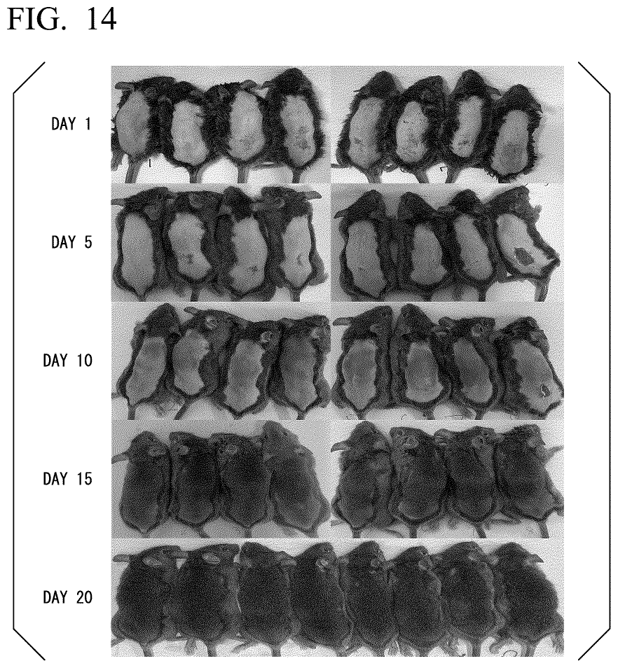

FIG. 14 A photograph showing a process of hair growth of a mouse in a control group in Experimental Example 5.

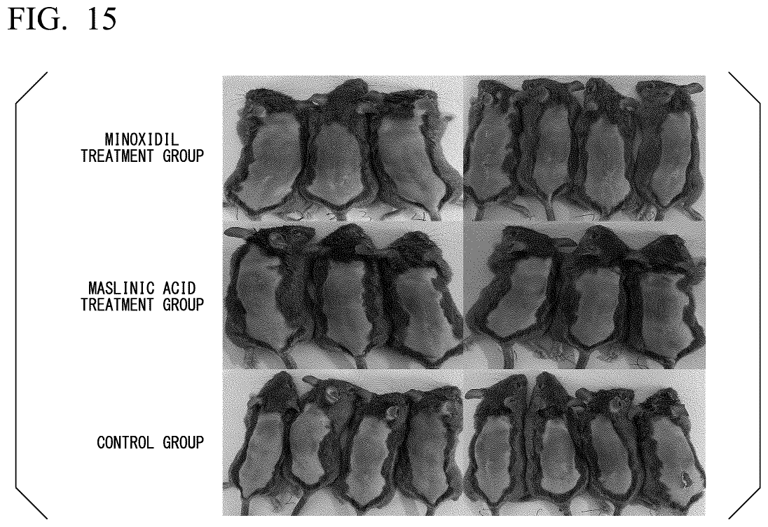

FIG. 15 A photograph of the dorsal skin of a mouse in each group on the 10th day in Experimental Example 5.

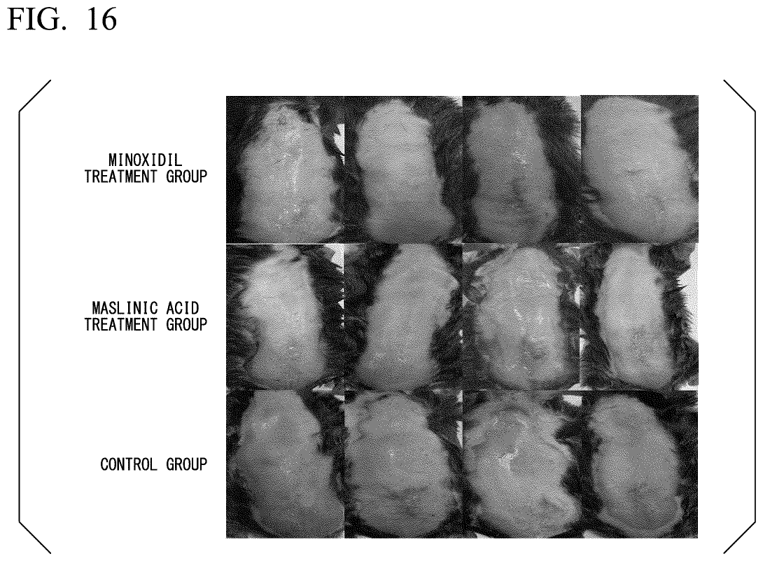

FIG. 16 A photograph of the dorsal skin of a mouse in each group on the 20th day in Experimental Example 5.

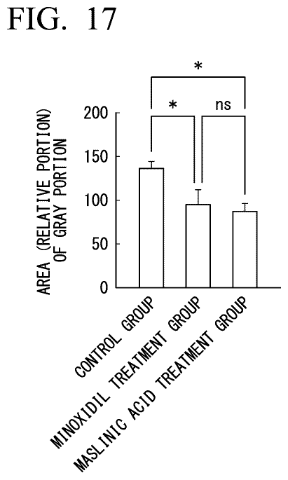

FIG. 17 A graph showing the measurement results of an area of a gray portion (catagen) on the dorsal skin of a mouse in each group on the 20th day in Experimental Example 5.

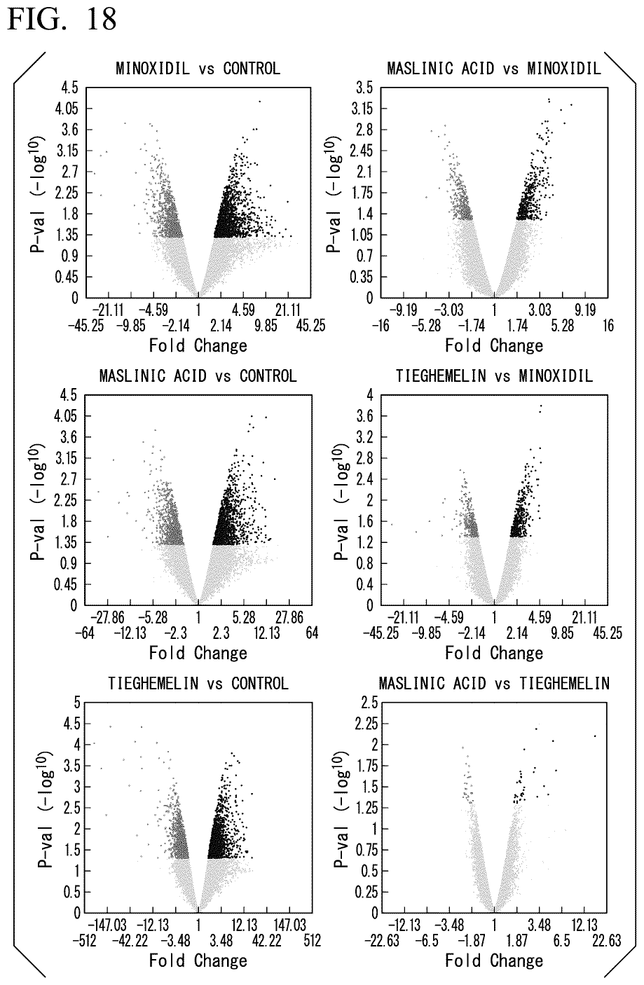

FIG. 18 A volcano plot showing the results of comparing transcriptomes in mouse-derived samples in each group in Experimental Example 6.

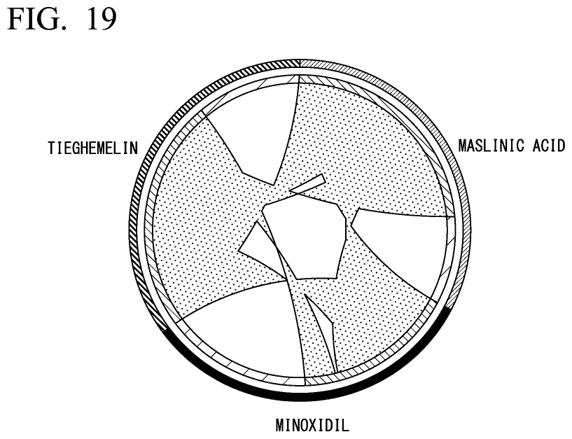

FIG. 19 A circos plot created based on expressed genes in each group in Experimental Example 6.

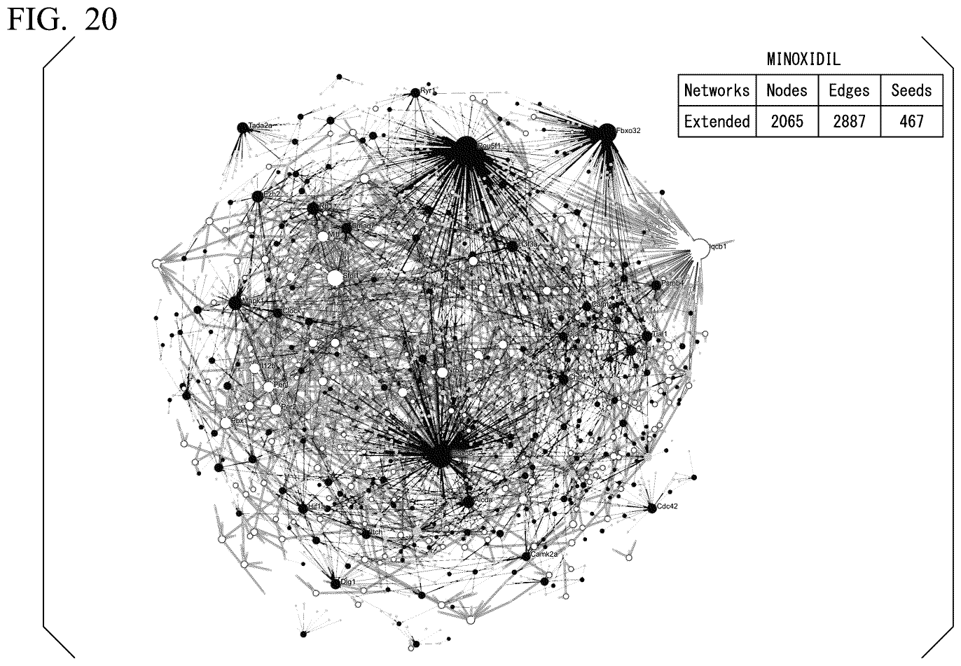

FIG. 20 A view showing the results of extended network analysis in Experimental Example 8.

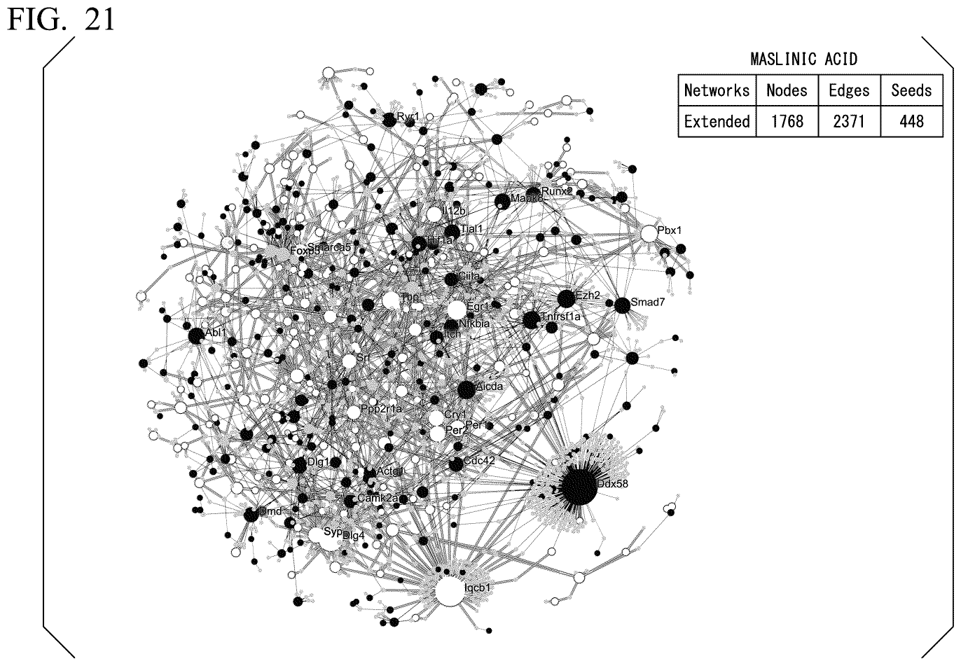

FIG. 21 A view showing the results of extended network analysis in Experimental Example 8.

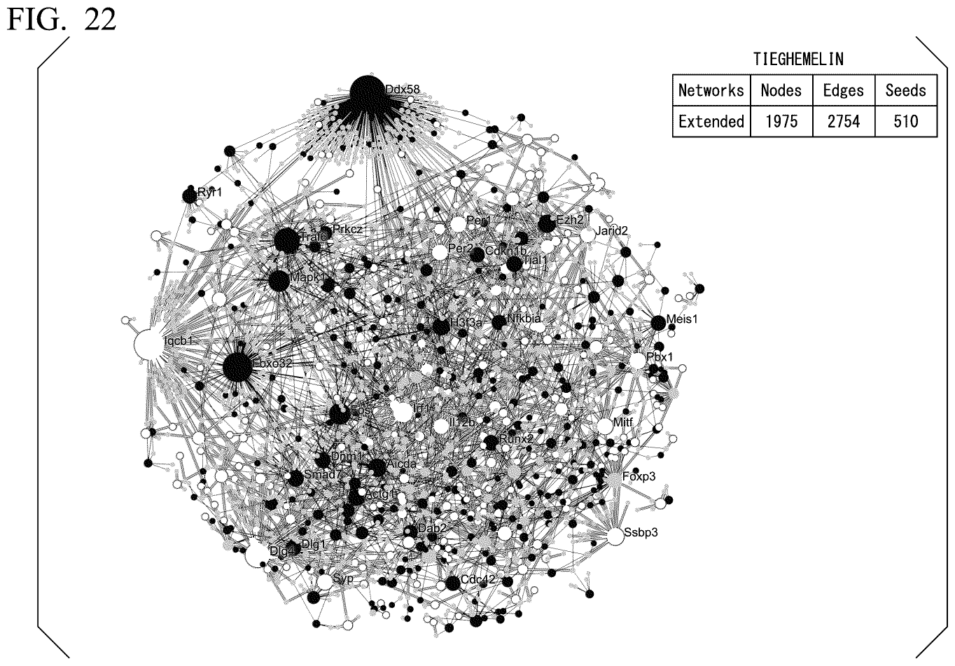

FIG. 22 A view showing the results of extended network analysis in Experimental Example 8.

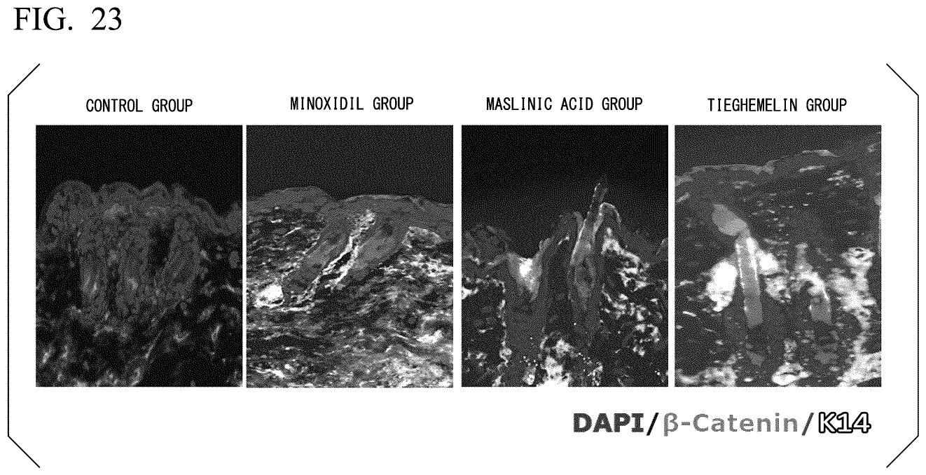

FIG. 23 A photomicrograph showing the results of detecting the expressions of β-catenin and K14 by fluorescence immunostaining in Experimental Example 10.

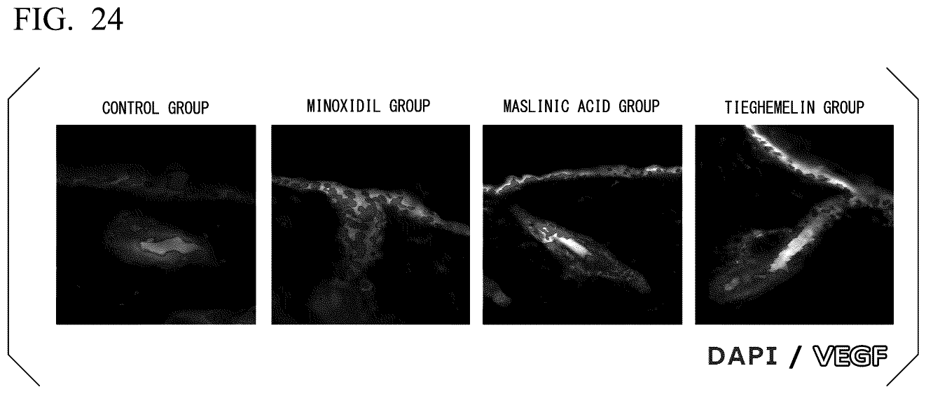

FIG. 24 A photomicrograph showing the results of detecting the expression of VEGF by fluorescence immunostaining in Experimental Example 10.

### DESCRIPTION OF EMBODIMENTS

### [Notation of Gene Names and Protein Names]

In the present specification, human genes and human proteins are represented by uppercase alphabets. Furthermore, a first letter of a mouse gene shall be represented by an uppercase alphabet, and subsequent letters shall be represented by a lowercase alphabet. In addition, a mouse protein shall be represented by an uppercase alphabet. However, in some cases, human genes, mouse genes, human proteins, and mouse proteins may be described without being strictly distinguished from each other.

### [Agent for Inducing Cilium]

In one embodiment, the present invention provides an agent for inducing cilium having an oleanane-type triterpenoid or a glycoside thereof as an active ingredient. In the present specification, the oleanane-type triterpenoid means a compound having a pentacyclic oleanane skeleton and a compound similar to the compound. In addition, the glycoside of an oleanane-type triterpenoid means a saponin having the oleanane-type triterpenoid as an aglycone.

As described later in Examples, the present inventors have found that both the oleanane-type triterpenoid and a glycoside of the oleanane-type triterpenoid increase the expression level of IQCB1, which is a gene inducing formation of cilium, and activate hair papilla cells, hair matrix cells, or the like, thereby having a hair loss preventing effect, a hair growth promoting effect, an hair cycle regulating effect, or the like.

As described later in Examples, the present inventors have found that the presence or absence of a sugar chain does not give a significant influence on the activation of hair papilla cells, hair matrix cells, and the like. Both the oleanane-type triterpenoid and a glycoside of the oleanane-type triterpenoid strongly induce formation of cilium. From the results of the gene analysis, the present inventors have also found that the oleanane-type triterpenoid and a glycoside thereof have a very high similarity in action.

The agent for inducing cilium of the present embodiment can be referred to as an activator of hair papilla cells or hair matrix cells, a hair growth promoter, or the like.

In the agent for inducing cilium of the present embodiment, the oleanane-type triterpenoid or a glycoside thereof is preferably a compound represented by Formula (A) or (B).

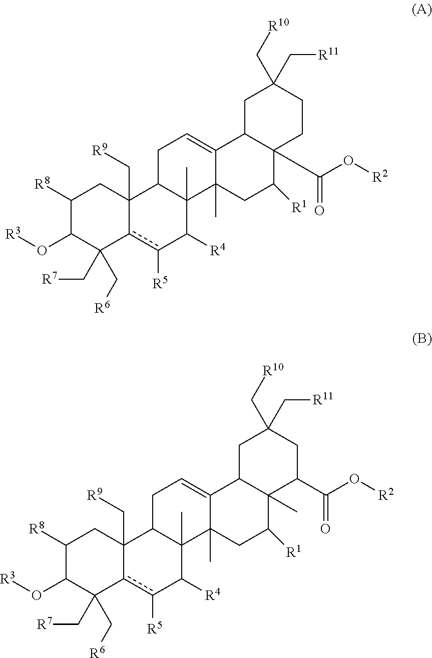

In Formulae (A) and (B), R¹, R⁴, R⁵, R⁶, R⁷, R⁸, R⁹, R¹⁰, and R¹¹ each independently represent a hydrogen atom or a hydroxyl group, R² and R³ each independently represent a hydrogen atom or an oligosaccharide chain including 1 to 5 monosaccharide units, and [symbol]
represents a single bond or a double bond].

In the agent for inducing cilium of the present embodiment, the compound represented by Formula (A) or (B) may be a compound represented by Formula (1).

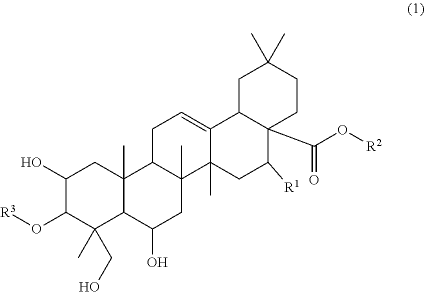

In Formula (1), the definitions of R¹, R², and R³ are the same as those in Formula (A) or (B). That is, R¹'s each independently represent a hydrogen atom or a hydroxyl group, and R² and R³ each independently represent a hydrogen atom or an oligosaccharide chain including 1 to 5 monosaccharide units. Here, the oligosaccharide chain including 1 to 5 monosaccharide units means an oligosaccharide chain formed by bonding 1 to 5 molecules of monosaccharides. From the viewpoint of exhibiting the effects of the invention of the present application, the number of monosaccharide units of R² is preferably 3 to 5, and more preferably 4 or 5. From the viewpoint of exhibiting the effects of the invention of the present application, the number of monosaccharide units of R³ is preferably 1 to 3, and more preferably 1 or 2.

The “hair growth cycle” is also called a “hair cycle”, and consists of three phases of an anagen, a catagen, and a telogen in a hair follicle. In the anagen, a dermal papilla cell at the proximal end of the hair follicle regulates the induction of a hair cycle and the formation of a hair shaft. Subsequently, the hair follicle undergoes a catagen and then transitions to a telogen in which the hair is shed.

As a cause of the hair loss in the head, a change in the hair growth cycle (a rapid transition from an anagen to a telogen, the extension of a telogen, and the like) is known. Therefore, such changes can be normalized and the hair loss can be suppressed by accelerating the hair growth cycle.

In the present specification, “accelerating the hair growth cycle” means that by an action of the oleanane-type triterpenoid or a glycoside thereof, the formation of cilium is induced and the period of each phase constituting the hair growth cycle is shortened, as compared with a case where the compound is not allowed to act.

Whether or not the hair growth cycle is accelerated can be determined by measuring the proliferation rate of dermal papilla cells of human hair follicles or the expression level of a hair growth marker.

The cilium is involved in the morphogenesis of hair follicles in the hair cycle during the embryonic stage and after birth. This function is through a Sonic Hedgehog (SHH) pathway, and is related to KIF3A and IFT88, which are two important cilium-forming genes.

As described later in Examples, the present inventors have found that the agent for inducing cilium of the present embodiment significantly upregulates the IQCB1 gene. It has been reported that the IQCB1 gene plays an important role in formation of cilium. In addition, it has been reported that the mutation of the IQCB1 gene is correlated with dysregulation, a kidney disease, and a retinal disease.

However, the fact that the IQCB1 gene is involved in the activation of hair papilla cells, the activation of hair matrix cells, hair growth, and the like has not been known in the related art, and has been revealed for the first time by the present inventors.

In the present specification, the “hair growth promoting effect” means an effect of improving a hair growth function (for example, an increase in the number of times a hair growth cycle is repeated) due to the acceleration of the hair growth cycle. In addition, the “hair loss preventing effect” means an effect of preventing hair loss or hair thinning due to the acceleration of the hair growth cycle. In addition, the “hair cycle regulating effect” means an effect of normalizing or improving a hair growth cycle due to the acceleration of the hair growth cycle. Examples of “normalizing or improving the hair growth cycle” include suppression of a rapid transition from an anagen to a telogen, and normalization or improvement of the extension of a telogen.

In the agent for inducing cilium of the present embodiment, R² in Formula (A) or (B) can be a group represented by any one of Formulae (2) to (7). As a result, the effects of the invention of the present application can be further exhibited.

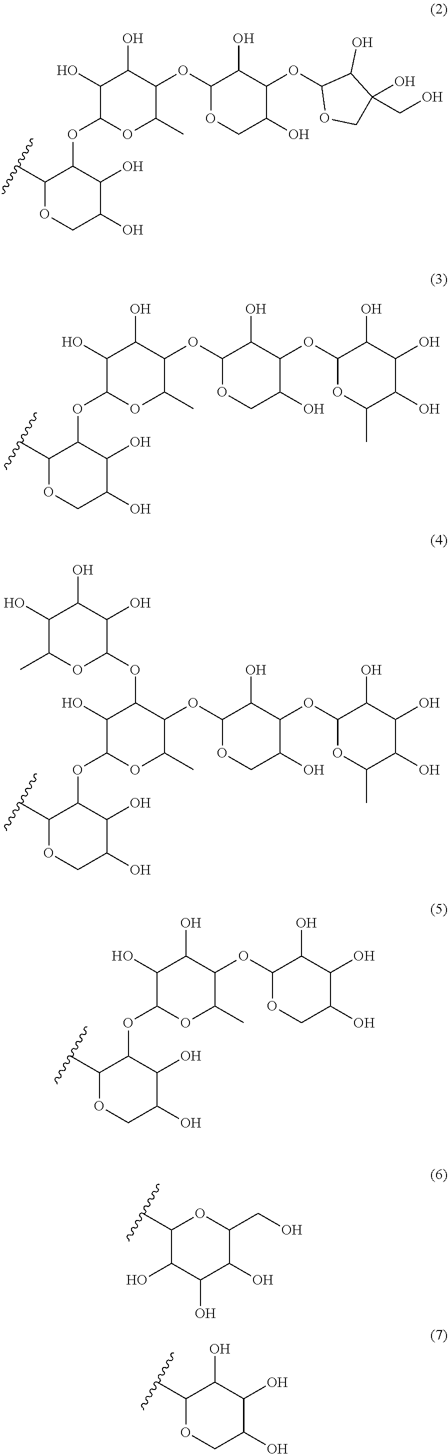

In the agent for inducing cilium of the present embodiment, R³ in Formula (A) or (B) can be a group represented by any one of Formulae (8) to (12). As a result, the effects of the invention of the present application can be further exhibited.

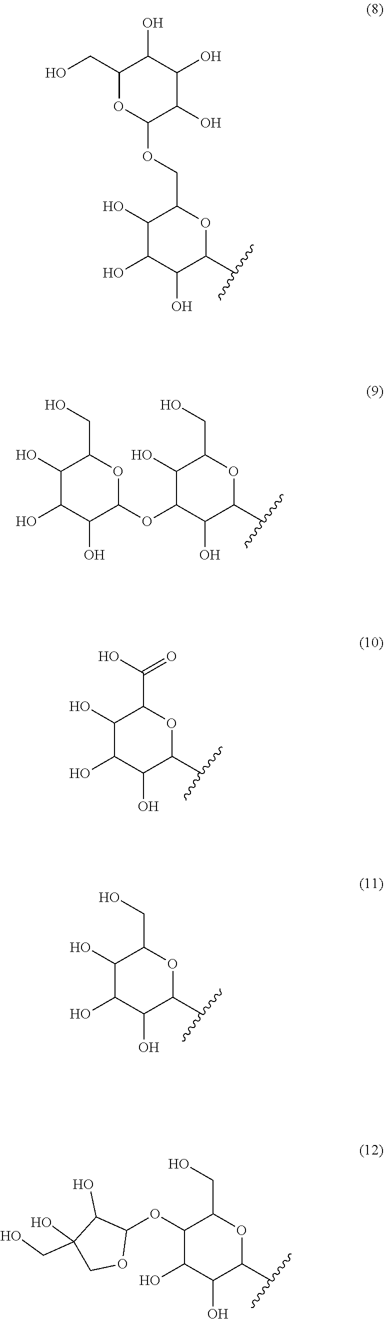 [SMILES: *C1OC(COC2OC(CO)C(O)C(O)C2O)C(O)C(O)C1O ; *C1OC(CO)C(O)C(OC2OC(CO)C(O)C(O)C2O)C1O ; *C1OC(C(=O)O)C(O)C(O)C1O ; *C1OC(CO)C(O)C(O)C1O ; *C1OC(CO)C(OC2OCC(O)(CO)C2O)C(O)C1O]

As an example of the method for introducing the compound represented by Formula (A) or (B), a method of extracting from a plant of the family Sapotaceae is exemplified. Examples of the plant of the family Sapotaceae include Argania Spinosa and Mimusops Elengi.

More specific examples of the oleanane-type triterpenoid represented by Formula (A) or (B) include compounds represented by any one of Formulae (13) to (41). In addition, more specific examples of the glycoside of the oleanane-type triterpenoid represented by Formula (A) or (B) include compounds represented by any one of Formulae (42) to (59) and (P6). As a result, the effects of the invention of the present application can be further exhibited. In Formulae (13) to (59) and (P6), the compound name and the CAS number are added for the compounds that can be specified.

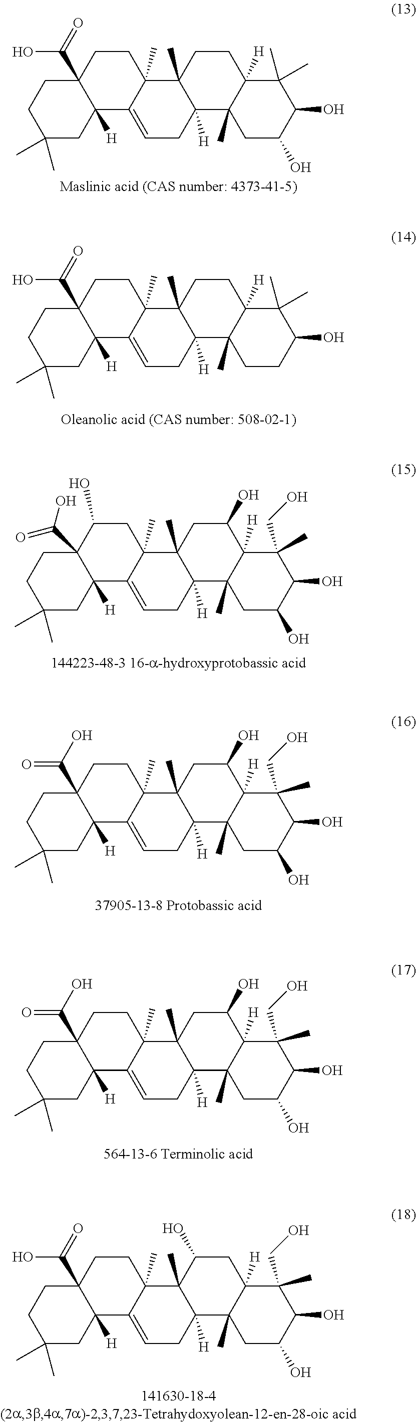

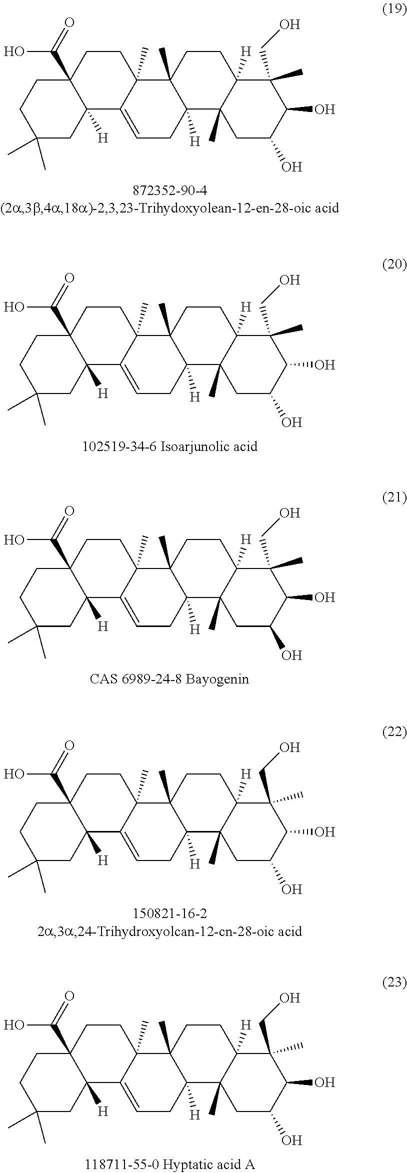

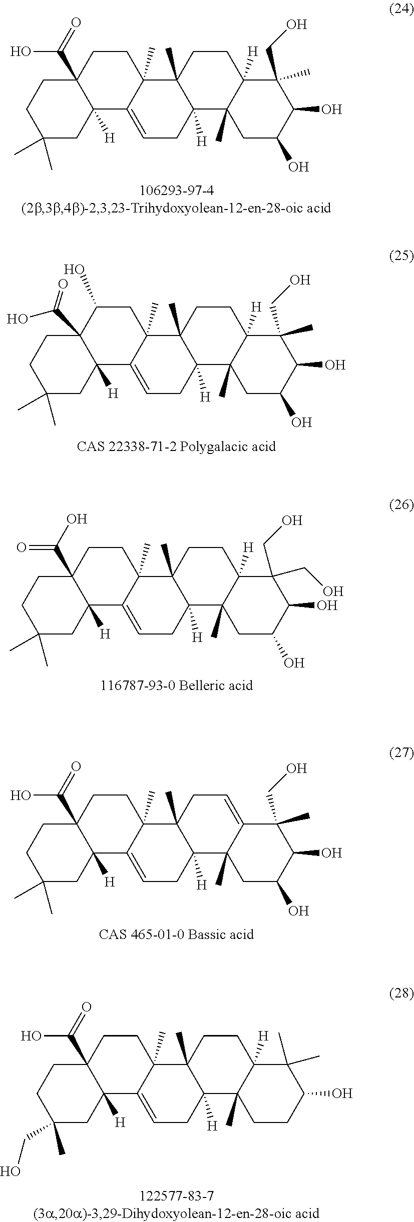

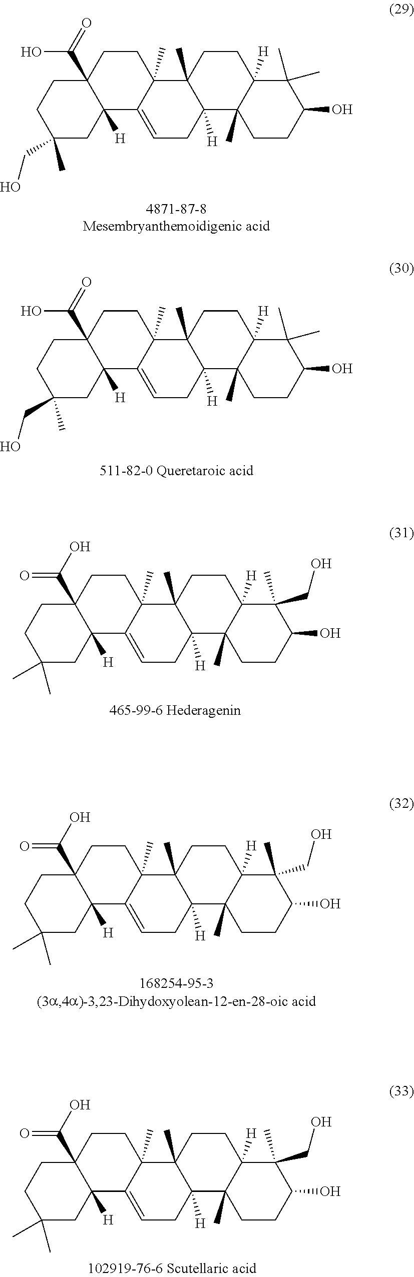

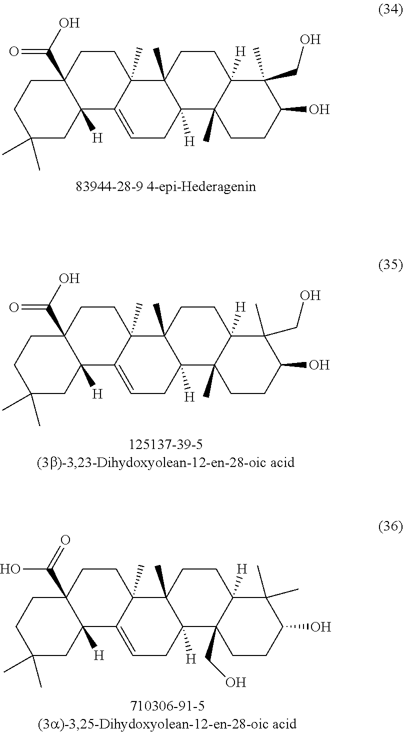 [SMILES: CC1(C)CC[C@]2(C(=O)O)CC[C@]3(C)C(=CC[C@@H]4[C@@]5(C)CC[C@H](O)[C@](C)(CO)[C@@H]5CC[C@]43C)[C@@H]2C1 ; CC1(C)CC[C@]2(C(=O)O)CC[C@]3(C)C(=CC[C@@H]4[C@@]5(C)CC[C@H](O)C(C)(CO)[C@@H]5CC[C@]43C)[C@@H]2C1 ; CC1(C)CC[C@]2(C(=O)O)CC[C@]3(C)C(=CC[C@@H]4[C@@]5(CO)CC[C@@H](O)C(C)(C)[C@@H]5CC[C@]43C)[C@@H]2C1]

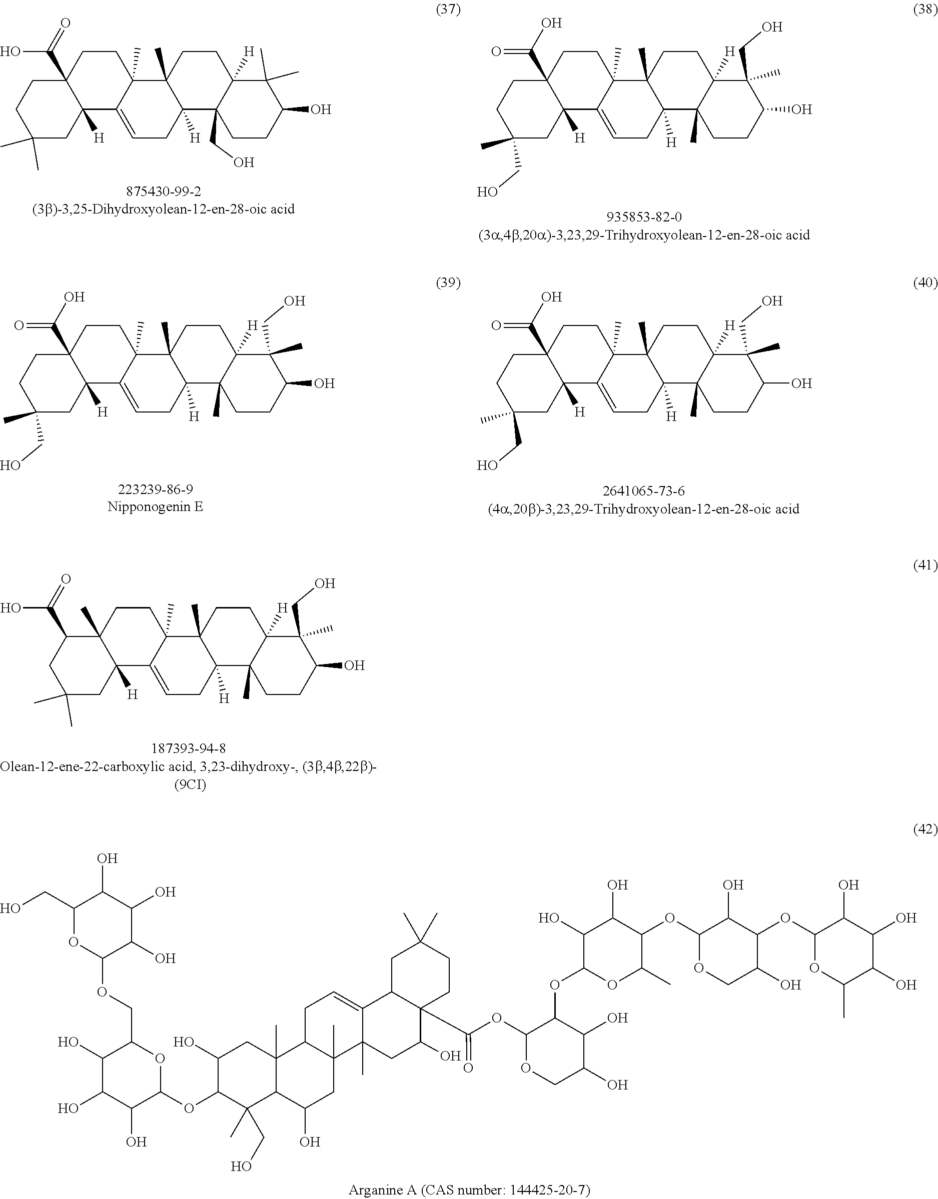

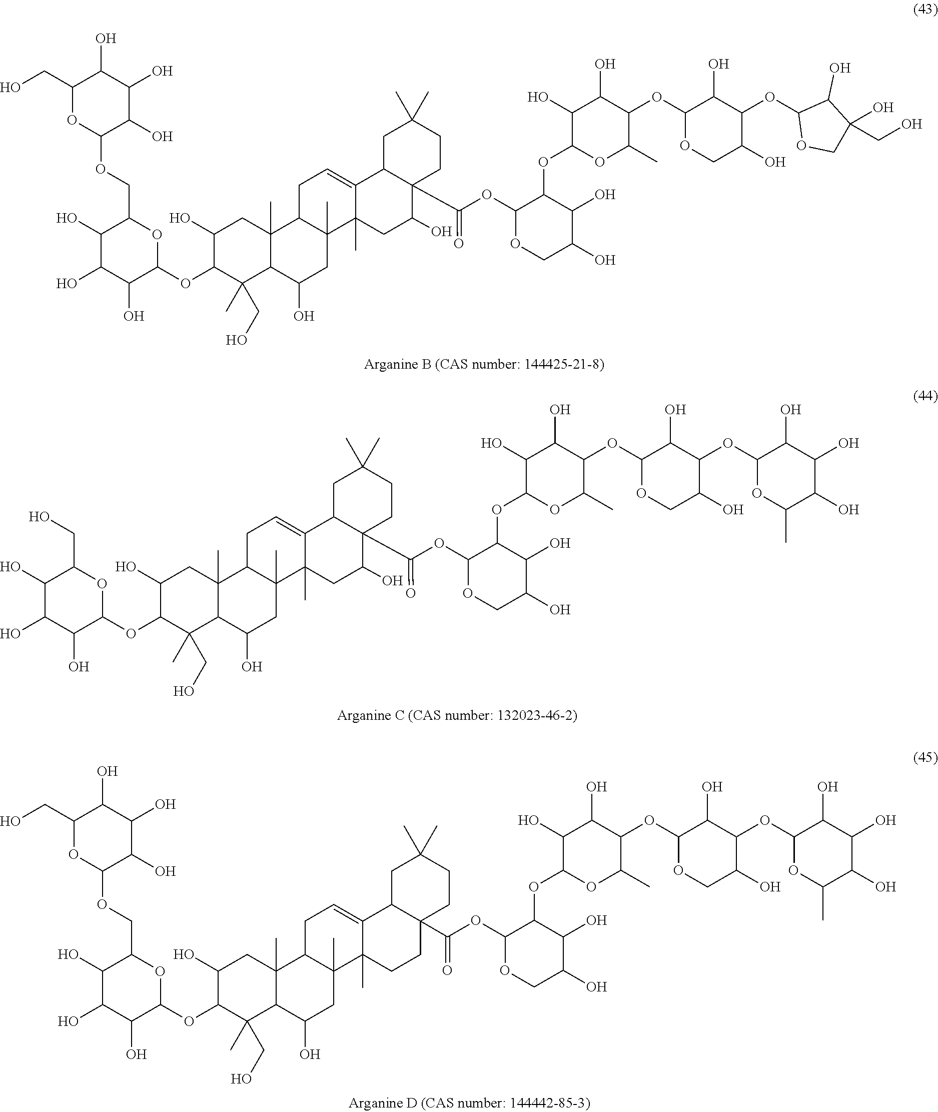

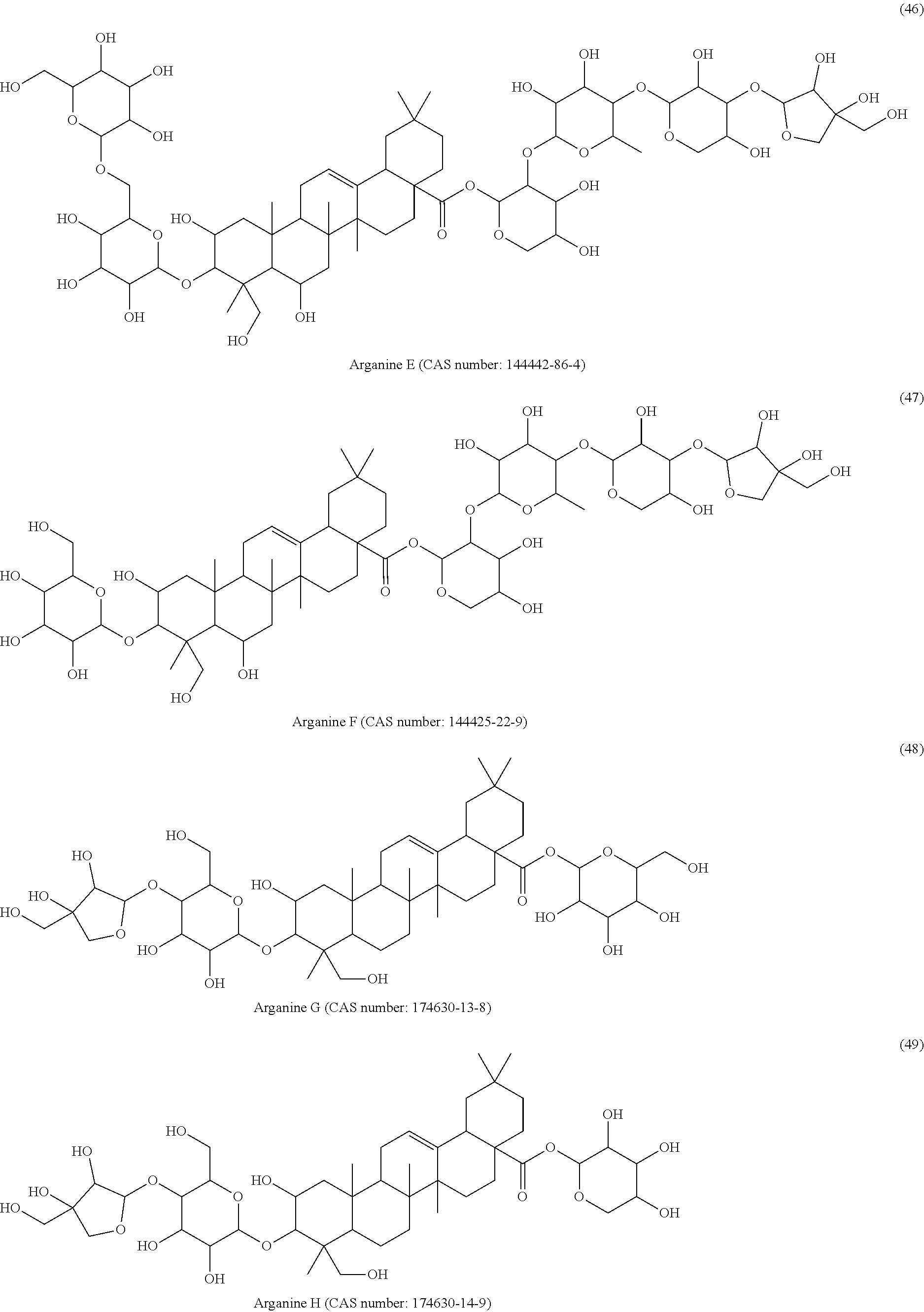

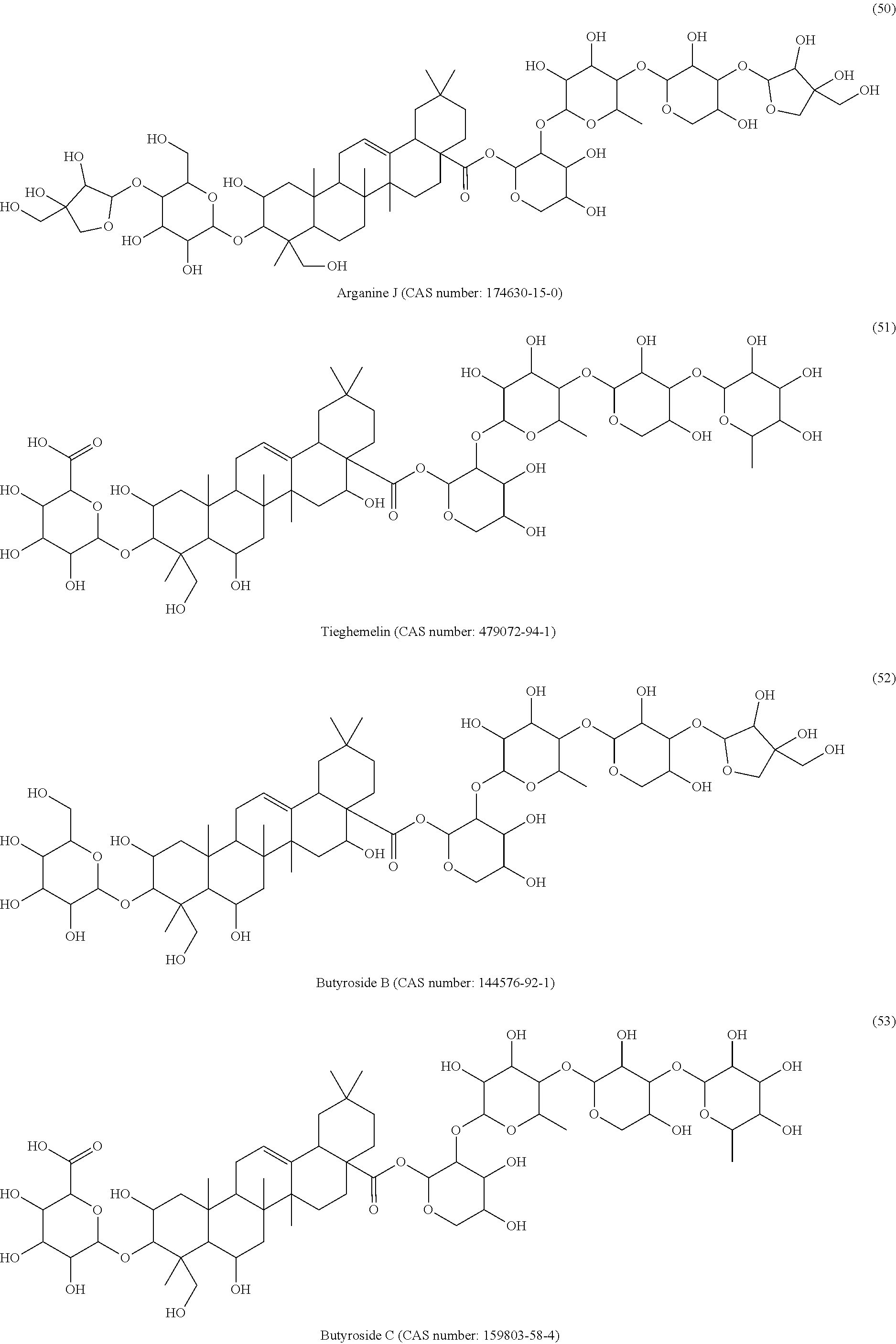

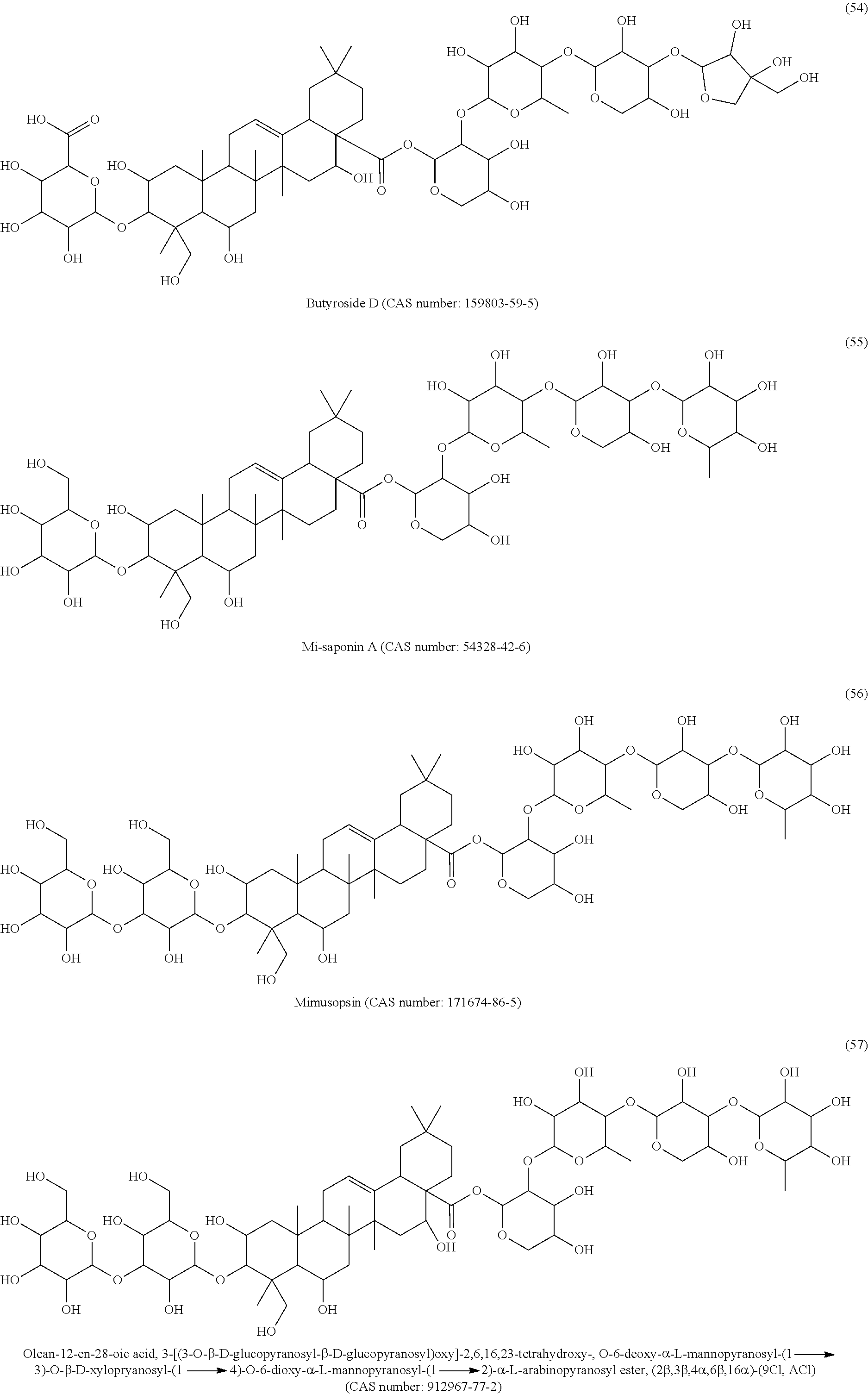

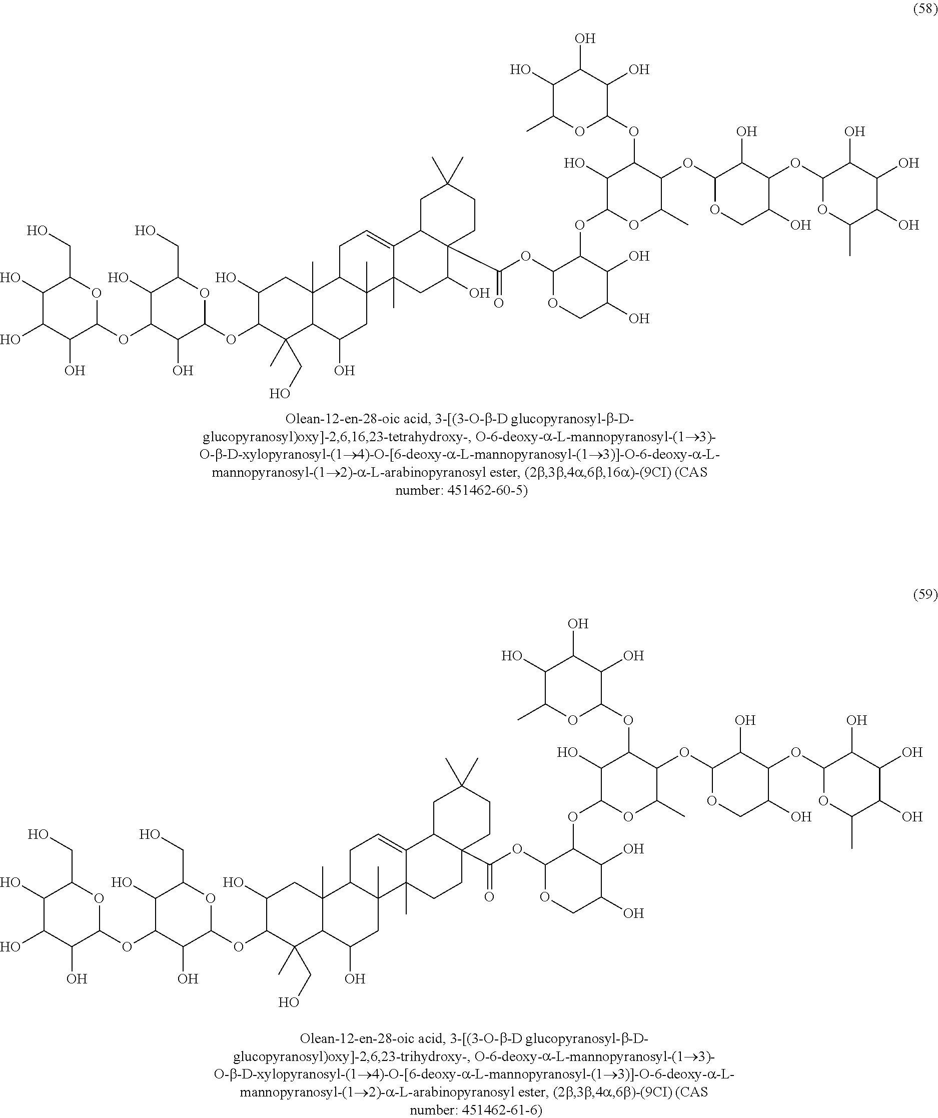

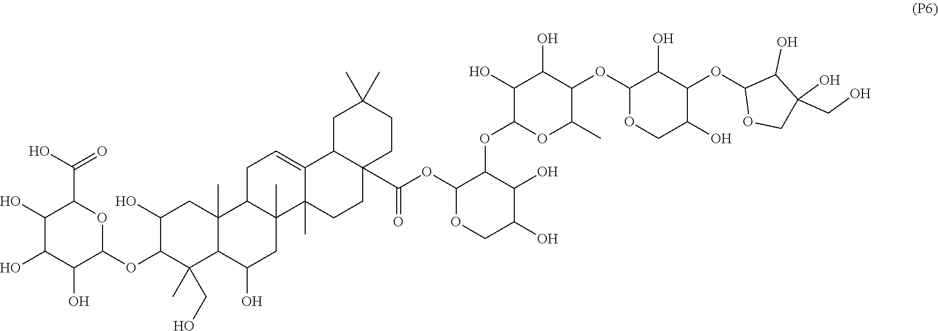

In the agent for inducing cilium of the present embodiment, the compound represented by Formula (A) or (B) is preferably a compound selected from the group consisting of maslinic acid, oleanolic acid, 16α-hydroxyprotobassic acid, protobassic acid, arganine A, arganine B, arganine C, arganine D, arganine E, arganine F, arganine G, arganine H, arganine J, tieghemelin, butyroside B, butyroside C, butyroside D, Mi-saponin A, mimusopsin, olean-12-en-28-oic acid, 3-[(3-O-β-D-glucopyranosyl-β-D-glucopyranosyl)oxy]-2,6,16,23-tetrahydroxy-, O-6-deoxy-α-L-mannopyranosyl-(1→3)-O-β-D-xylopyranosyl-(1→4)-O-6-deoxy-α-L-mannopyranosyl-(1→2)-α-L-arabinopyranosyl ester, (2β,3β,4α,6β,16α)-(9CI, ACI), olean-12-en-28-oic acid, 3-[(3-O-β-D-glucopyranosyl-β-D-glucopyranosyl)oxy]-2,6,16,23-tetrahydroxy-, O-6-deoxy-α-L-mannopyranosyl-(1→3)-O-3-D-xylopyranosyl-(1→4)-O-[6-deoxy-α-L-mannopyranosyl-(1→3)]-O-6-deoxy-α-L-mannopyranosyl-(1→2)-α-L-arabinopyranosyl ester, (2β,3β,4α,6β,16α)-(9CI), olean-12-en-28-oic acid, 3-[(3-O-β-D-glucopyranosyl-β-D-glucopyranosyl)oxy]-2,6,23-trihydroxy-, O-6-deoxy-α-L-mannopyranosyl-(1→3)-O-β-D-xylopyranosyl-(1→4)-O-[6-deoxy-α-L-mannopyranosyl-(1→3)]-O-6-deoxy-α-L-mannopyranosyl-(1→2)-α-L-arabinopyranosyl ester, (2β,3β,4α,6β)-(9CI), and a compound represented by Formula (P6). As a result, the effects of the invention of the present application can be further exhibited.

In the agent for inducing cilium of the present embodiment, the compound represented by Formula (A) or (B) is more preferably a compound selected from the group consisting of butyroside D, arganine D, arganine E, tieghemelin, maslinic acid, and Mi-saponin A. As a result, the effects of the invention of the present application can be further exhibited. As described later in Examples, these compounds have been confirmed to have an effect of activating IQCB1 that induces formation of cilium and activating hair papilla cells or hair matrix cells.

The agent for inducing cilium of the present embodiment may be for oral administration. As described later in Examples, the agent for inducing cilium of the present embodiment can permeate intestinal epithelial cells from the apex side (lumen side) to the basal side (vascular side). These results show that the agent for inducing cilium of the present embodiment can exhibit the effects by oral administration.

The agent for inducing cilium of the present embodiment is preferably formulated as a composition for inducing cilium, which includes the above-mentioned agent for inducing cilium and a pharmaceutically acceptable carrier. As a result, the effects of the invention of the present application can be further exhibited. The composition for inducing cilium can be referred to as a composition that activates hair papilla cells or hair matrix cells. The composition for inducing cilium activates IQCB1 that induces formation of cilium, and thus serves as a trigger for activating a series of genes.

The pharmaceutically acceptable carrier is not particularly limited, and examples thereof include an excipient, a binder, a disintegrating agent, a lubricant, an emulsifier, a thickener, a wetting agent, and a solvent for an injection agent. In addition, the composition for inducing cilium may further include an additive.

The additive is not particularly limited, and examples thereof include a preservative, a pH adjuster, a stabilizer, an ultraviolet absorber, an antioxidant, a colorant, a fragrance, and a cellulose nanofiber. The cellulose nanofiber is a fiber composed of a cellulose, the fiber width (fiber diameter (equivalent circle diameter)) is usually 1 nm to 500 nm, and the fiber length is usually 0.1 m to 6 km.

As the pharmaceutically acceptable carrier and the additive, for example, general raw materials described in the Japanese Pharmacopoeia, 16th edition, and the like can be used.

Examples of the dosage form of the composition for inducing cilium include orally administrable dosage forms such as a tablet, a coated tablet, a pill, a powder, a granular agent, a capsule agent, a liquid agent, a suspension, and an emulsion, and parenterally administrable dosage forms such as an injection agent, a suppository, and a topical skin preparation.

Examples of the topical skin preparation include dosage forms such as a cream, a lotion, a skin toner, an emulsion, a foundation, a pack agent, a foam agent, a plaster agent, an ointment agent, a patch agent, and an aerosol agent. The topical skin preparation may be the emulsion. The emulsion means a dispersion-based solution in which both a dispersed substance and a dispersion medium are liquids.

The emulsion may be an oil-in-water type emulsion (an emulsion composed of a water phase as a continuous phase and oil droplets dispersed in the water phase) or a water-in-oil type emulsion (an emulsion composed of an oil phase as a continuous phase and water droplets dispersed in oil), but is preferably the oil-in-water type emulsion. In addition, the physical properties can be suitably adjusted by adding cellulose nanofibers to the emulsion.

By forming the composition for inducing cilium in the form of an emulsion, it is possible to improve the absorbability of the oleanane-type triterpenoid or a glycoside thereof in a case where the composition is applied to a living body.

The composition for inducing cilium may be a therapeutic drug for alopecia or may be a therapeutic drug that activates hair papilla cells or hair matrix cells. That is, the composition for inducing cilium may be a pharmaceutical composition. Alternatively, the composition for inducing cilium may be a cosmetic or a food such as a supplement.

A content of the hair growth agent (the compound represented by Formula (1)) in the composition for inducing cilium is, for example, in a range of 0.01% to 50% by mass, 0.01% to 30% by mass, 0.01% to 10% by mass, 0.01% to 5% by mass, or 0.01% to 1% by mass in terms of a solid content (dry weight) of the hair growth agent.

A method for administering the agent for inducing cilium or the composition for inducing cilium is not particularly limited, and may be appropriately determined according to the symptom, the body weight, the age, the sex, or the like of a subject to be administered. For example, a tablet, a coated tablet, a pill, a powder, a granular agent, a capsule agent, a liquid agent, a suspension, or an emulsion is administered orally. In addition, the injection agent is administered singly or as a mixture with a general replacement fluid such as glucose and amino acids, and as necessary, an intraarterial, intramuscular, intradermal, subcutaneous, or intraperitoneal injection is administered. A suppository is rectally administered. A topical skin preparation is applied, affixed, or sprayed onto an affected area.

A dose of the agent for inducing cilium or the composition for inducing cilium varies depending on the symptom, the body weight, the age, the sex, or the like of a subject to be administered, and cannot be determined unconditionally. However, in a case of oral administration, for example, the active ingredient (the compound represented by Formula (1)) at 0.01 to 5,000 mg/kg body weight per day may be administered. In addition, in a case of the injection agent, for example, 0.01 to 500 mg per day of the active ingredient may be administered. Furthermore, in the case of a suppository, for example, 0.01 to 1,000 mg per day of the active ingredient may be administered. In addition, in a case of a topical skin preparation, for example, 0.01 to 500 mg per day of the active ingredient may be administered. As described later in Examples, the agent for inducing cilium or the composition for inducing cilium of the present embodiment can permeate intestinal epithelial cells from the apex side (lumen side) to the basal side (vascular side). These results show that the agent for inducing cilium of the present embodiment can exhibit the effects by oral administration. Therefore, the agent for inducing cilium or the composition for inducing cilium may be for oral administration.

### OTHER EMBODIMENTS

In one embodiment, the present invention provides a method for treating alopecia, the method including administering an effective amount of an oleanane-type triterpenoid or a glycoside thereof to a patient in need of a medical treatment.

In one embodiment, the present invention provides an oleanane-type triterpenoid or a glycoside thereof for use in a treatment of alopecia.

In one embodiment, the present invention provides a use of an oleanane-type triterpenoid or a glycoside thereof for producing a agent for inducing cilium.

In each of these embodiments, the oleanane-type triterpenoid or a glycoside thereof is the same as described above.

### EXAMPLES

Next, the present invention will be described in more detail with reference to Examples, but is not limited to the following Examples.

### Experimental Example 1

### (Examination on Activation of Hair Papilla Cells by Glycoside of Oleanane-Type Triterpenoid)

The activation effect by a glycoside of an oleanane-type triterpenoid was examined using dermal papilla cells of human hair follicles (HFDPCs) which are mesenchymal cells separated from the papilla of the scalp hair follicle of a normal human. As the glycoside of the oleanane-type triterpenoid (which may hereinafter be referred to as “saponin”), arganine D, arganine E, and butyroside D were used. A high proliferation rate of the HFDPCs is correlated with the acceleration of the hair growth cycle.

First, HFDPCs were seeded in a 96-well plate at 3×10⁵ cells/well and incubated at 37° C. in a humidified atmosphere of 5% CO₂. As a culture medium, a culture medium for hair papilla cell growth to which growth factors (a bovine fetal serum, an insulin, a transferrin, a triiodothyronine, a bovine pituitary extract, or a cyproterone solution) had been added was used.

After 24 hours, the culture medium was exchanged with a culture medium containing each of various concentrations of each saponin, and the cells were incubated for 48 hours.

Subsequently, the cell proliferation was measured using 3-(4,5-dimethyl-thiazol-2-yl)-2,5-diphenyltetrazolium bromide (MTT). Specifically, an MTT reagent (5 mg/mL) was added to the cells and incubated for 8 hours, then 10% sodium dodecyl sulfate (SDS) was added thereto, and the cells were incubated overnight. Thereafter, the absorbance at 570 nm was measured using a microplate reader, and the cell proliferation was quantified as the proportion (%) of the absorbance to an absorbance of the untreated cells. In addition, the survival rate of cells was also measured by a trypan blue exclusion method.

FIG. 1 is a graph showing the results of examining the influence of arganine D on the proliferation of HFDPCs. FIG. 2 is a graph showing the results of examining the influence of arganine E on the proliferation of HFDPCs. FIG. 3 is a graph showing the results of examining the influence of butyroside D on the proliferation of HFDPCs. In FIGS. 1 to 3, the values on the graphs indicate the average value±a standard deviation (n=3). In addition, “*” indicates that there is a significant difference at p<0.05, and “**” indicates that there is a significant difference at p<0.01. In FIGS. 1 to 3, the higher the numerical value on the vertical axis, the more activated the hair papilla cells and hair matrix cells are, leading to a faster hair growth cycle and a higher hair loss preventing effect or hair growth promoting effect.

As a result, it was found that the arganine D significantly promotes the proliferation of the HFDPCs at 2, 5, and 10 μM. Furthermore, it was found that the arganine E significantly promotes the proliferation of HFDPCs at 2 μM. In addition, it was found that the butyroside D significantly promotes the proliferation of HFDPCs at 2, 5, and 10 μM.

### Experimental Example 2

### (Examination of Activation Effect for Hair Papilla Cells by Butyroside D)

The activation effect by the butyroside D was examined using human hair follicle dermal papilla cells (HFDPCs). A high proliferation rate of the HFDPCs is correlated with the acceleration of the hair growth cycle.

First, HFDPCs were seeded in a 96-well plate at 3×10⁵ cells/well and incubated at 37° C. in a humidified atmosphere of 5% CO₂. As a culture medium, a culture medium for hair papilla cell growth to which growth factors (a bovine fetal serum, an insulin, a transferrin, a triiodothyronine, a bovine pituitary extract, or a cyproterone solution) had been added was used.

After 24 hours, the culture medium was exchanged with a culture medium containing each of 0, 0.5, 1, 2, 4, 8, and 10 nM butyroside D, and the cells were incubated for 48 hours.

Subsequently, an MTT reagent (5 mg/mL) was added to the cells and incubated for 8 hours, then 10% sodium dodecyl sulfate (SDS) was added thereto, and the cells were incubated overnight. Thereafter, the absorbance at 570 nm was measured using a microplate reader, and the cell proliferation was quantified as a proportion (%) of the absorbance to an absorbance of the untreated cells. In addition, the survival rate of cells was also measured by a trypan blue exclusion method.

FIG. 4 is a graph showing the results of examining the influence of butyroside D on the proliferation of HFDPCs. In FIG. 4, the values on the graphs indicate the average value±a standard deviation (n=3). In FIG. 4, the higher the numerical value of the vertical axis, the more activated the hair papilla cells and the hair matrix cells, leading to a faster hair growth cycle and a higher hair loss preventing effect or hair growth promoting effect.

### Experimental Example 3

### (Examination of Hair Growth Promoting Effect by Butyroside D)

The HFDPC was treated with butyroside D, and then the expression of the hair growth marker was confirmed by quantitative real-time RT-PCR.

As the hair growth markers, a CTNNB1 gene (β-catenin), an ALPL gene (alkaline phosphatase), and an FGF1 gene (fiblobrast growth factor 1) were examined. It is known that the β-catenin induces anagen of the hair cycle and promotes the proliferation of keratinocytes. ALPL is a representative hair growth marker in the hair papilla cells. FGF1 is a marker for morphogenesis of hair follicles.

HFDPCs were seeded in a 6-well plate at 5×10⁴ cells/well and incubated at 37° C. for 24 hours. Subsequently, the culture medium was removed and exchanged with a culture medium including 0 (Control), 0.5, 5, and 10 nM of butyroside D. In addition, for comparison, cells treated with a culture medium including minoxidil (0.1 μM), which is an existing pharmaceutical product, were also prepared.

Subsequently, after 48 hours, the cells were washed with cold PBS and a total RNA was extracted using an ISOGEN kit (Nippon Gene Co., Ltd.). Next, the total RNA was quantified using a NanoDrop 2000 spectrophotometer (Thermo Fisher Scientific) and quantitative real-time RT-PCR analysis was performed.

First, using a SuperScript III reverse transcription kit (Thermo Fisher Scientific), a cDNA was synthesized from the extracted total RNA by a cycling protocol of 95° C. for 10 minutes, 40 cycles of 95° C. for 15 seconds, and 60° C. for 1 minute.

Subsequently, a real-time PCR was performed using a 7500 Fast Real-Time PCR system (Software 1.3.1, Thermo Fisher Scientific) and TaqMan probes specific for CTNNB1, ALPL, and FGF1. GAPDH was used as an endogenous control and the relative mRNA amount was calculated by applying a 2^{-ΔΔ}Ct method by comparison with GAPDH.

FIG. 5 is a graph showing the results of quantitative real-time RT-PCR of the CTNNB1 gene. FIG. 6 is a graph showing the results of quantitative real-time RT-PCR of the ALPL gene. FIG. 7 is a graph showing the results of quantitative real-time RT-PCR of the FGF1 gene. In FIGS. 5 to 7, the values of the graphs indicate the average value±a standard deviation (n=3). In addition, “*” indicates that there is a significant difference at p<0.05, and “**” indicates that there is a significant difference at p<0.01.

As a result, it was found that the expression level of the CTNNB1 gene in HFDPC was significantly increased by the treatment with 5 nM and 10 nM butyroside D. In addition, the effect of increasing the expression level of the CTNNB1 gene was more than the effect of the treatment with 0.1 M minoxidil which had been used as a positive control.

Moreover, it was found that the expression level of the ALPL gene in the HFDPC was significantly increased by the treatment with 5 nM and 10 nM butyroside D. In addition, the effect of increasing the expression level of the ALPL gene was more than the effect of the treatment with 0.1 M minoxidil which had been used as a positive control.

Moreover, it was found that the expression level of the FGF1 gene in the HFDPC was significantly increased by the treatment with 0.5 nM, 5 nM, and 10 nM butyroside D. In addition, the effect of increasing the expression level of the ALPL gene was more than the effect of the treatment with 0.1 μM minoxidil which had been used as a positive control.

The results above indicate that butyroside D has a cilium-induction promoting effect.

### Experimental Example 4

### (Examination Using Co-Culture System of Caco-2 and HFDPC)

An examination was carried out using a co-culture system of Caco-2 and an HFDPC which are human intestinal epithelial cell lines. FIG. 8 is a schematic view illustrating a co-culture system of the present Experimental Example. As shown in FIG. 8, Caco-2 cells were seeded inside a 96-well insert and HFDPCs were seeded on a receiver plate. A permeable membrane having a pore size of 0.8 m was disposed on the bottom surface of the 96-well insert. According to this co-culture system, it is possible to examine whether or not a drug is permeable to the intestinal epithelial cells, that is, whether or not the drug can exhibit the effects by oral administration.

In a case where the drug is permeable to the intestinal epithelial cells, with the addition of the drug into the inside of the transwell insert of the co-culture system, the drug permeates from the apex side (lumen side) to the basal side (vascular side) of the Caco-2 cells. Furthermore, the drug that permeated acts on the HFDPCs seeded on the receiver plate.

A mixture in which each saponin was mixed at a ratio shown in Table 1 below (which may hereinafter be referred to as a “saponin mixture”) was used as a sample for the examination. The saponin mixture was added to the inside of the transwell insert of the co-culture system so that a concentration thereof was 5 μg/mL. Subsequently, the expression of the hair growth marker in the HFDPCs seeded on the receiver plate was measured by quantitative real-time PCR after 2 hours, 6 hours, and 12 hours.

TABLE 1

| Saponin | Blending amount (parts by mass) |
|---|---|
| Arganine A | 30 |
| Arganine B | 20 |
| Tieghemelin | 20 |
| Butyroside D | 10 |
| Arganine D | 3 |
| Arganine E | 2 |
| Butyroside C | 10 |
| Compound represented | 5 |
| by Formula (P6) | |

As the hair growth markers, a CTNNB1 gene (β-catenin), an ALPL gene (alkaline phosphatase), and an FGF1 gene (fiblobrast growth factor 1) were measured. It is known that the β-catenin induces anagen of the hair cycle and promotes the proliferation of keratinocytes. ALPL is a representative hair growth marker in the hair papilla cells. FGF1 is a marker for morphogenesis of hair follicles.

Moreover, for comparison, a sample in which instead of the saponin mixture, a pharmaceutical finasteride was added to the inside of the transwell insert of the co-culture system so that the concentration thereof was 10 μM was prepared and subjected to the same measurement. In addition, as a control, a sample to which the drug had not been added was prepared, and the same measurement was performed.

FIGS. 9A to 9C are graphs showing the measurement results of the expression level of the β-catenin gene after 2 hours, 6 hours, and 12 hours, respectively, from the addition of the drug. FIGS. 10A to 10C are graphs showing the measurement results of the expression level of the ALPL gene after 2 hours, 6 hours, and 12 hours, respectively, from the addition of the drug. FIGS. 11A to 11C are graphs showing the measurement results of the expression level of the FGF1 gene after 2 hours, 6 hours, and 12 hours, respectively, from the addition of the drug. In FIGS. 9A to 11C, “*”, “**”, “***”, and “****” indicate that there is a significant difference with respect to the control at p<0.05, p<0.01, p<0.001, and p<0.0001, respectively.

As a result, it was shown that the finasteride and the saponin mixture induced the expression of the hair growth marker regardless of the treatment time. These results show that the saponin can exhibit the hair loss preventing effect or the hair growth promoting effect even in a case where the saponin is orally administered.

In addition, it was found that the effect of increasing the expression of the 3-catenin gene and the FGF1 gene by the administration of the saponin mixture was higher than the effect of increasing the expression by finasteride.

Moreover, since the saponin mixture exhibited an oral hair growth effect in a short period of time (2 hours), a high permeation absorption in the intestinal epithelium, the skin, or the like was suggested.

### Experimental Example 5

### (Examination 1 of Hair Growth Promoting Effect In Vivo)

The oleanane-type triterpenoid was applied to the dorsal skin of a mouse and the hair growth promoting effect was examined. Maslinic acid was used as the oleanane-type triterpenoid. In addition, for comparison, a group in which minoxidil was used and a control group in which only a solvent was applied were prepared.

FIG. 12 is a schematic view showing an experimental schedule. After acclimatization of a C3H male mouse, the dorsal skin was depilated with a shaver and a depilatory cream. Thereafter, 150 μL of 1% minoxidil (n=8), 1% maslinic acid (n=6), or a solvent (n=8) was applied to the mouse in each group every day for 20 days, and the mouse was observed over time. No influence on the body weight of the mouse during the experimental period was observed.

It is known that the hair cycle of a mouse has three stages of an anagen, a catagen, and a telogen. During the anagen (the 1st day to the 16th day), the hair follicle grows into the deep subcutaneous part. In the catagen (the 17th day to the 19th day), the hair follicle stops growing and regresses to the upper subcutaneous part. In the telogen (the 20th day to the 29th day), the hair shaft is in a state where it can be lost at any time. It is known that the color of the dorsal skin of a mouse changes to pink, gray, and black due to the hair cycle. FIG. 13 is a view showing a change in the color of the dorsal skin of a mouse due to the hair cycle.

FIG. 14 is a photograph showing a process of hair growth of a mouse in a control group. As a result, it was found that the control group transitioned from the telogen to the anagen on the 10th day. According to the same observation, the transition to the late anagen was observed on the 10th day in the minoxidil treatment group. The results show that the hair cycle was promoted by the administration of minoxidil.

In addition, in the maslinic acid treatment group, the transition to the late anagen was observed on the 10th day in the same manner as in the minoxidil treatment group. These results show that the hair cycle was promoted by the administration of the maslinic acid.

FIG. 15 is a photograph of the dorsal skin of a mouse in each group on the 10th day. As a result, the speed of a transition from the telogen to the anagen was in the following order.
Minoxidil≈Maslinic acid>Control group

Moreover, it was observed that the minoxidil treatment group and the maslinic acid treatment group had a larger black area indicating the late anagen than the control group. These results show that minoxidil and maslinic acid have a hair growth promoting effect.

FIG. 16 is a photograph of the dorsal skin of a mouse in each group on the 20th day. FIG. 17 is a graph showing the measurement results of the area of the gray portion (catagen) on the dorsal skin of a mouse in each group on the 20th day. In FIG. 17, “ns” indicates that there is no significant difference and “*” indicates that there is a significant difference at p<0.05.

As a result, it was observed that the minoxidil treatment group and the maslinic acid treatment group transitioned to the next telogen. The speed of transition to the telogen after the anagen and the catagen was in the following order.
Minoxidil=Maslinic acid>Control group

### Experimental Example 6

### (Examination 2 of Hair Growth Promoting Effect In Vivo)

A mouse in each group of Experimental Example 5 and a mouse in the group which had been subjected to the same treatment as in Experimental Example 5 using 1% tieghemelin (n=6) were euthanized on the 20th day, and the dorsal skin was collected and subjected to transcriptome analysis.

FIG. 18 is a volcano plot showing the results of comparing transcriptomes in mouse-derived samples in each group. Many of the differentially expressed genes showed 2- to 5-fold changes in expression level. In addition, it was found that the differentially expressed genes in the dorsal skins of the mice showed the same pattern between the maslinic acid treatment group and the tieghemelin treatment group.

FIG. 19 is a Circos plot created based on the expressed genes in each group. The Circos plot shows how the expressed genes overlap each other. The genes that overlap among the three groups are shown in the gray region, and the genes that are specifically expressed are shown in the white region.

### Experimental Example 7

### (Examination 3 of Hair Growth Promoting Effect In Vivo)

Gene ontology (GO) analysis and GO enrichment analysis were performed based on the results of the transcriptome analysis in Experimental Example 6.

The number of genes with increased expression that are common among the minoxidil treatment group, the maslinic acid treatment group, and the tieghemelin treatment group was 342. Many of these were related to tissue morphology formation, angiogenesis control, and stimulus responsiveness.

One-hundred-twenty-eight genes with increased expression common between the minoxidil treatment group and the tieghemelin treatment group were related to a CD40-controlled MAPK cascade and a protein phosphorylation function.

Eighty-one genes with increased expression common between the minoxidil treatment group and the maslinic acid treatment group were related to control responses of stimulus responsiveness and DNA damage.

Moreover, the biological process in which genes with increased expression were enriched was related to a stimulus response, a developmental process, and a signal transduction (mainly an ERK1, an ERK2 cascade, an MAPK cascade, and a Notch signaling pathway).

The number of genes with decreased expression common among the minoxidil treatment group, the maslinic acid treatment group, and the tieghemelin treatment group was 316. These were related to cell differentiation and translation initiation.

Ninety-four genes with decreased expression common between the minoxidil treatment group and the tieghemelin treatment group were related to p53 or an equivalent mediator involved in a cell cycle, DNA damage, and cell proliferation.

Seventy-two genes with decreased expression common between the minoxidil treatment group and the maslinic acid treatment group were related to the regulation of a kinase activity.

In addition, the biological process in which genes with decreased expression were enriched was related to a metabolic control such as fatty acid or oxidative phosphorylation, and a signal transduction (mainly a negative control of an MAPK or TOR signal, and a protein kinase B signal).

Subsequently, term enrichment analysis of the genes with expression increased specifically with respect to the skin tissue was performed. Using tissue-specific expression analysis (TSEA), genes related to the skin tissue were filtered, and functions that were specifically enriched in the maslinic acid treatment group and the tieghemelin treatment group and were not enriched in the minoxidil treatment group were selected.

As a result, it was found that the maslinic acid treatment group was enriched with functions related to skin barriers, keratinocytes, proliferation of epithelial cells, pigmentation, and interactions between cytokines and cytokine receptors.

In addition, it was found that the functions such as the proliferation of keratinocytes and epithelial cells, the control of the MAPK cascade, the intercellular adhesion, and the activation of the cytokine receptor interaction were enriched in the tieghemelin treatment group.

### Experimental Example 8

### (Examination 4 of Hair Growth Promoting Effect In Vivo)

Extended network analysis was performed based on the results of the transcriptome analysis in Experimental Example 6. FIGS. 20 to 22 are views showing the results of the extended network analysis. FIG. 20 is an analysis result obtained from a dataset of the minoxidil treatment group, FIG. 21 is an analysis result obtained from a dataset of the maslinic acid treatment group, and FIG. 22 is an analysis result obtained from a dataset of the tieghemelin treatment group. In FIGS. 20 to 22, a white circle indicates a gene with increased expression, and a black circle indicates a gene with decreased expression. In addition, the size of the circle indicates the magnitude of a change in expression level.

As a result, it was found that the number of species of the expanded protein-protein interaction (PPI) networks obtained from the dataset of the minoxidil treatment group, the maslinic acid treatment group, and the tieghemelin treatment group was 400 or more.

According to the analysis of the gene list in each group, it was found that a large network obtained from DDX58, IQCB1, and the other related proteins is included. DDX58 is an innate immune receptor that senses viral nucleic acids in the cytoplasm and activates a downstream signal transduction cascade leading to the production of type I interferons or inflammatory cytokines. IQCB1 is a protein known to be involved in formation of cilium.

Iqcb1 was upregulated by the minoxidil treatment, the maslinic acid treatment, and tieghemelin treatment. In addition, it was found that the expression level of Iqcb1 was increased more in the maslinic acid treatment group and the tieghemelin treatment group than in the minoxidil treatment group. The involvement of Iqcb1 in the hair growth has not previously been reported, and was revealed for the first time by the present inventors.

It has been reported that IQCB1 interacts with CEP290 to regulate formation of cilium. In addition, it has been suggested that CEP290 interacts with KIF3A and IFT88 to promote formation of cilium and interacts with the SHH pathway.

The role of the IQCB1 gene in hair growth has not been reported in the related art. According to the results of the present experiment, it was for the first time shown that the IQCB1 gene is upregulated by the maslinic acid treatment and the tieghemelin treatment, and is involved in the hair follicle cycle.

IQCB1 interacts with CEP290 to adjust formation of cilium, and CEP290 interacts with IFT88 and KIF3A to promote formation of cilium and further interacts with the SHH pathway. As a result, it is considered to promote the activation of hair papilla cells or hair matrix cells. That is, the IQCB1 gene is the most upstream gene that serves as a trigger for activating hair papilla cells or hair matrix cells.

Since the Iqcb1 gene was upregulated in all of the minoxidil administration group, the triterpenoid (maslinic acid) administration group, and the triterpenoid glycoside (tieghemelin) administration group, which are positive controls, it was shown that the Iqcb1 gene plays an important role in the activation of hair papilla cells or hair matrix cells.

In addition, the Iqcb1 gene was expressed more strongly in the maslinic acid administration group and the tieghemelin administration group than in the minoxidil administration group, indicating that maslinic acid and tieghemelin have a higher effect of activating hair papilla cells or hair matrix cells than minoxidil.

### Experimental Example 9

### (Examination 5 of Hair Growth Promoting Effect In Vivo)

A fluctuation in expression of the hair cycle-related genes was examined, based on the results of the transcriptome analysis in Experimental Example 6.

As a result, it was found that the expression of NF-κB was decreased in the maslinic acid treatment group and the tieghemelin treatment group. NF-κB is a catagen-related marker and is also an inflammation promoting marker.

In addition, it was found that the expression level of BMP4, which promotes the telogen, was decreased in the maslinic acid treatment group. This suggests that the maslinic acid promotes the differentiation of the outer sheath of the hair shaft, which is essential for hair growth.

In addition, it was found that the expression level of Aepb1, which is a regulatory factor for the hair follicle in the telogen, was decreased in the tieghemelin treatment group. This suggests that the tieghemelin delayed the transition of the hair follicle to the telogen and extended the anagen.

Moreover, it was found that the expression level of KRT15, which is a marker of the telogen, was decreased in the tieghemelin treatment group. The expression of KRT15 is involved in the preparation for the hair follicle entering the telogen. This suggests that the transition from the telogen to the anagen is delayed by the tieghemelin treatment.

### Experimental Example 10

### (Examination 6 of Hair Growth Promoting Effect In Vivo)

A mouse in each group of Experimental Examples 5 and 6 was euthanized on the 20th day, the dorsal skin was collected, and the tissue section was subjected to immunostaining.

FIG. 23 is a photomicrograph showing the results of detecting the expressions of β-catenin and K14 by fluorescence immunostaining. In addition, the nuclei were stained with 4′,6-diamidino-2-phenylindole (DAPI). The β-catenin is a hair growth promoting marker and is also a marker for the start of the anagen. K14 is an epidermal marker and is known to increase with the differentiation of hair follicle stem cells.

As a result, it was found that the effect of the maslinic acid treatment is the same as that of the minoxidil treatment, and the increase in the expression of K14 and 3-catenin induces a rapid transition to the anagen.

Furthermore, from the shape of the hair follicle, it was shown that the transition to a stage of the late anagen (IV), in which the anagen has already been induced and the anagen has almost ended, has been made.

In the tieghemelin treatment group, K14 and β-catenin were highly expressed. From the shape of the hair follicle, it was suggested that the hair follicle was at the stage of the anagen (II), the anagen was extended, and the growth of the hair was continued.

FIG. 24 is a photomicrograph showing the results of detecting the expression of VEGF by fluorescence immunostaining. In addition, the nuclei were stained with DAPI. VEGF is known to improve the vascularization of hair follicles, increase the size of hair follicles and hair, and promote the growth and circulation of hair follicles.

As a result, it was found that in the maslinic acid treatment group and the minoxidil treatment group, the expression of VEGF was increased, and the sizes of the hair follicle and the hair shaft were increased. It was also found that the VEGF is highly expressed in the tieghemelin treatment group. It was suggested that the tieghemelin treatment induce the extension of the anagen by increasing the size of the hair follicle due to the VEGF stimulation.

### Experimental Example 11

### (Preparation of Emulsion Including Glycoside of Oleanane-Type Triterpenoid)

Emulsions of Examples 1 to 9 including a glycoside of an oleanane-type triterpenoid were prepared with compositions shown in Table 2 below. In addition, an emulsion of Comparative Example 1, which did not include a glycoside of an oleanane-type triterpenoid, was also prepared. By forming the compound in the form of an emulsion, the absorbability of the oleanane-type triterpenoid can be improved in a case of being applied to a living body.

TABLE 2

| | Parts by mass | | | | | | | | | |
|---|---|---|---|---|---|---|---|---|---|---|
| Name of | Example | Example | Example | Example | Example | Example | Example | Example | Example | Comparative |
| component | 1 | 2 | 3 | 4 | 5 | 6 | 7 | 8 | 9 | Example 1 |
| Tieghemelin | 0.3 | 0.3 | 0.3 | 0.3 | 0.3 | — | — | — | — | — |
| Butyroside D | — | — | — | — | — | 0.3 | — | — | — | — |
| Mi-saponin A | — | — | — | — | — | — | 0.3 | 0.1 | 0.3 | — |
| Soybean | — | — | — | — | — | — | — | — | — | 0.3 |
| saponin | | | | | | | | | | |
| Cellulose | 3 | 3 | 3 | 3 | 3 | 3 | 3 | 3 | — | 3 |
| nanofiber | | | | | | | | | | |
| Mineral oil | 5 | — | — | — | — | — | — | — | — | — |
| (liquid | | | | | | | | | | |
| paraffin) | | | | | | | | | | |
| Olive oil | — | 5 | — | — | — | 5 | 5 | 5 | 5 | 5 |
| Squalane | — | — | 5 | — | — | — | — | — | — | — |
| Argan oil | — | — | — | 5 | — | — | — | — | — | — |
| Shea better | — | — | — | — | 5 | — | — | — | — | — |
| Distilled | 91.7 | 91.7 | 91.7 | 91.7 | 91.7 | 91.7 | 91.7 | 91.9 | 94.7 | 91.7 |
| water | | | | | | | | | | |

### INDUSTRIAL APPLICABILITY

According to the present invention, it is possible to provide a compound having an effect of inducing formation of cilium and activating hair papilla cells or hair matrix cells.

1-10. (canceled)

11. A method for treating alopecia, comprising:
administering an effective amount of an oleanane-type triterpenoid or a glycoside thereof to a patient in need of a medical treatment.

12. The method according to claim 11,
wherein the oleanane-type triterpenoid or a glycoside thereof is a compound represented by the following Formula (A) or (B), 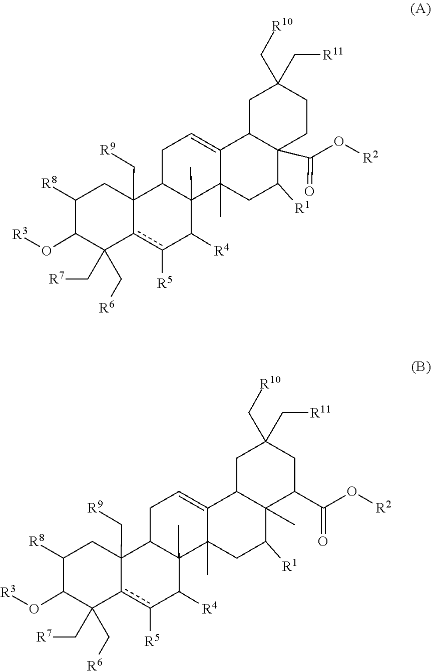
wherein in the Formulae (A) and (B), R¹, R⁴, R⁵, R⁶, R⁷, R⁸, R⁹, R¹⁰, and R¹¹ each independently represent a hydrogen atom or a hydroxyl group, R² and R³ each independently represent a hydrogen atom or an oligosaccharide chain including 1 to 5 monosaccharide units, and [symbol]
represents a single bond or a double bond.

13. The method according to claim 12,
wherein the oleanane-type triterpenoid or a glycoside thereof is a compound represented by the following Formula (1), 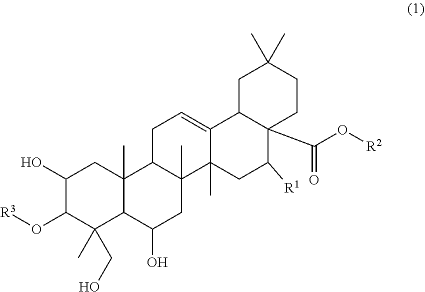
wherein in the Formula (1), R¹, R², and R³ each have the same definitions as those in the Formula (A) or (B).

14. The method according to claim 12,
wherein R² in the Formula (A) or (B) is a group represented by any one of the following Formulae (2) to (7),

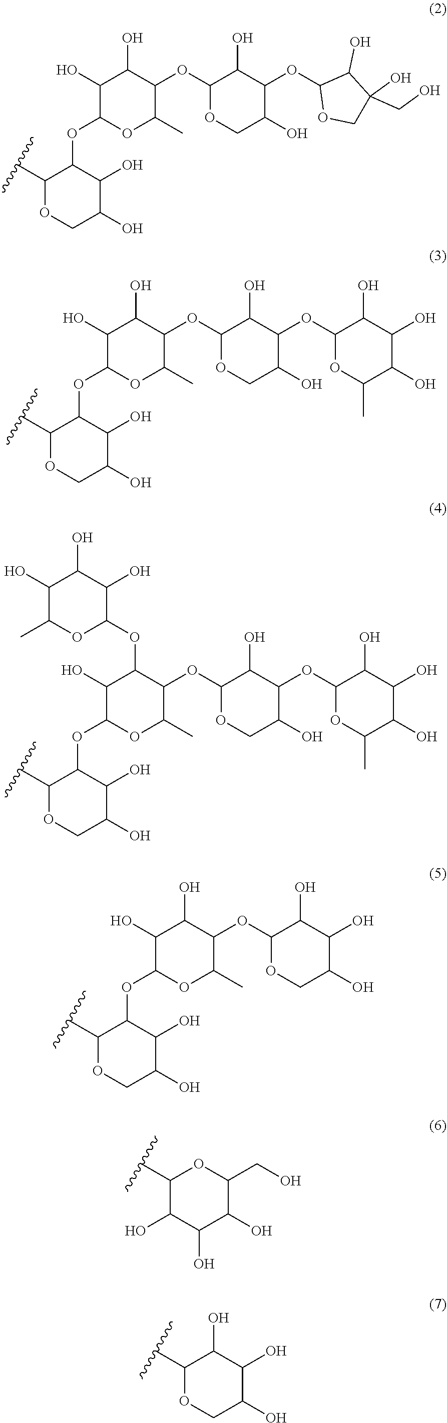 [SMILES: *C1OCC(O)C(O)C1OC1OC(C)C(OC2OCC(O)C(OC3OCC(O)(CO)C3O)C2O)C(O)C1O ; *C1OCC(O)C(O)C1OC1OC(C)C(OC2OCC(O)C(OC3OC(C)C(O)C(O)C3O)C2O)C(O)C1O ; *C1OCC(O)C(O)C1OC1OC(C)C(OC2OCC(O)C(OC3OC(C)C(O)C(O)C3O)C2O)C(OC2OC(C)C(O)C(O)C2O)C1O ; *C1OCC(O)C(O)C1OC1OC(C)C(OC2OCC(O)C(O)C2O)C(O)C1O ; *C1OC(CO)C(O)C(O)C1O ; *C1OCC(O)C(O)C1O]

15. The method according to claim 12,
wherein R³ in the Formula (A) or (B) is a group represented by any one of Formulae (8) to (12),

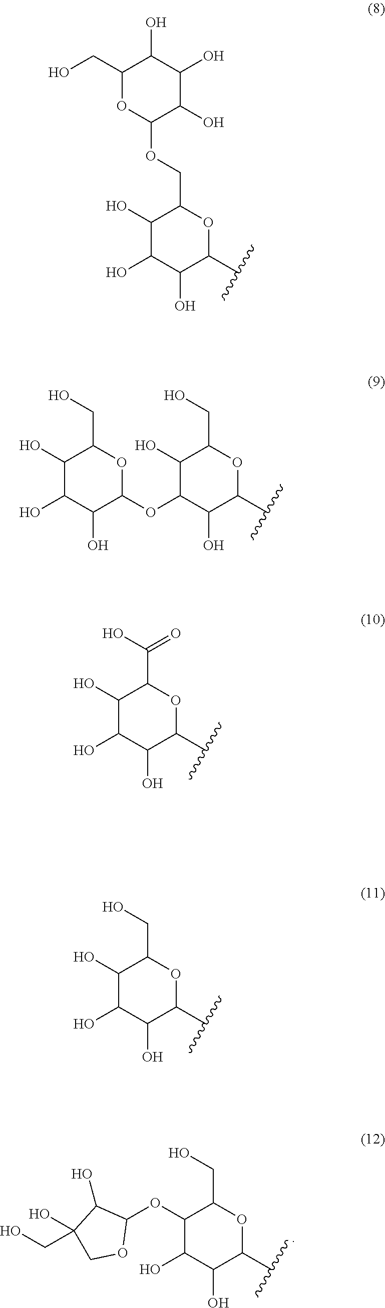 [SMILES: *C1OC(COC2OC(CO)C(O)C(O)C2O)C(O)C(O)C1O ; *C1OC(CO)C(O)C(OC2OC(CO)C(O)C(O)C2O)C1O ; *C1OC(C(=O)O)C(O)C(O)C1O ; *C1OC(CO)C(O)C(O)C1O ; *C1OC(CO)C(OC2OCC(O)(CO)C2O)C(O)C1O]

16. The method according to claim 12,
wherein the compound represented by the Formula (A) or (B) is a compound selected from the group consisting of maslinic acid (CAS number: 4373-41-5), oleanolic acid (CAS number: 508-02-1), 16α-hydroxyprotobassic acid (CAS number: 144223-48-3), protobassic acid (CAS number: 37905-13-8), arganine A (CAS number: 144425-20-7), arganine B (CAS number: 144425-21-8), arganine C (CAS number: 132023-46-2), arganine D (CAS number: 144442-85-3), arganine E (CAS number: 144442-86-4), arganine F (CAS number: 144425-22-9), arganine G (CAS number: 174630-13-8), arganine H (CAS number: 174630-14-9), arganine J (CAS number: 174630-15-0), tieghemelin (CAS number: 479072-94-1), butyroside B (CAS number: 144576-92-1), butyroside C (CAS number: 159803-58-4), butyroside D (CAS number: 159803-59-5), Mi-saponin A (CAS number: 54328-42-6), mimusopsin (CAS number: 171674-86-5), olean-12-en-28-oic acid, 3-[(3-O-β-D-glucopyranosyl-β-D-glucopyranosyl)oxy]-2,6,16,23-tetrahydroxy-, O-6-deoxy-α-L-mannopyranosyl-(1→3)-O-3-D-xylopyranosyl-(1→4)-O-6-deoxy-α-L-mannopyranosyl-(1→2)-α-L-arabinopyranosyl ester, (2β,3β,4α,6β,16α)-(9CI,ACI) (CAS number: 912967-77-2), olean-12-en-28-oic acid, 3-[(3-O-β-D-glucopyranosyl-β-D-glucopyranosyl)oxy]-2,6,16,23-tetrahydroxy-, O-6-deoxy-α-L-mannopyranosyl-(1→3)-O-β-D-xylopyranosyl-(1→4)-O-[6-deoxy-α-L-mannopyranosyl-(1→3)]-O-6-deoxy-α-L-mannopyranosyl-(1→2)-α-L-arabinopyranosyl ester, (2β,3β,4α,6β,16α)-(9CI) (CAS number: 451462-60-5), olean-12-en-28-oic acid, 3-[(3-O-β-D-glucopyranosyl-β-D-glucopyranosyl)oxy]-2,6,23-trihydroxy-, O-6-deoxy-α-L-mannopyranosyl-(1→3)-O-β-D-xylopyranosyl-(1→4)-O-[6-deoxy-α-L-mannopyranosyl-(1→3)]-O-6-deoxy-α-L-mannopyranosyl-(1→2)-α-L-arabinopyranosyl ester, (2β,3β,4α,6β)-(9CI) (CAS number: 451462-61-6), and a compound represented by the following Formula (P6),

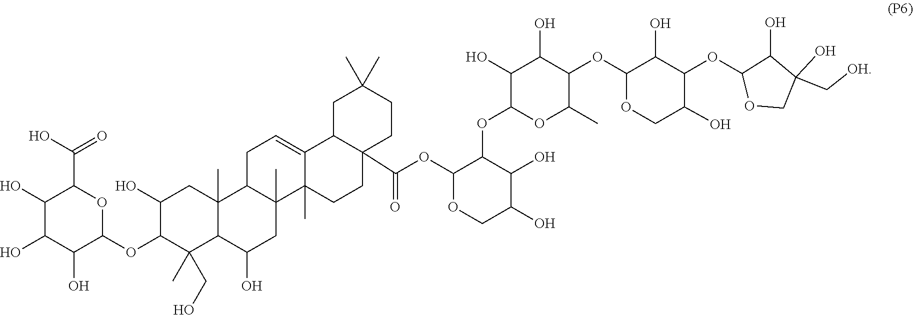

17. The method according to claim 12,
wherein the compound represented by the Formula (A) or (B) is a compound selected from the group consisting of butyroside D, arganine D, arganine E, tieghemelin, maslinic acid, and Mi-saponin A.

18. The method according to claim 11,
wherein the oleanane-type triterpenoid or a glycoside thereof is administered by oral administration.

19. The method according to claim 11,
wherein the oleanane-type triterpenoid or a glycoside thereof forms a composition with a pharmaceutically acceptable carrier.

20. The method according to claim 19,
wherein the composition is administered by oral administration.
